(19) United States
(12) Patent Application Publication
(10) Pub. No.: US 2023/0178234 A1
(43) Pub. Date: Jun. 8, 2023

(54) System and Method for Tracking Injection Site Information

(71) Applicant: Embecta Corp., Andover, MA (US)

(72) Inventors: Ryan Francis Bedell, Andover, MA (US); Danielle V. Butler, Andover, MA (US); Linda Charlite-Ruiz, Andover, MA (US); Douglas McClure, Andover, MA (US); Rita Saltiel-Berzin, Andover, MA (US); Sean M. Ulrich, Andover, MA (US); Joshua Daniel Coyle, Andover, MA (US); Alice Leung, Andover, MA (US); Teresa Oliveria, Andover, MA (US)

(73) Assignee: Embecta Corp., Andover, MA (US)

(21) Appl. No.: 18/161,550

(22) Filed: Jan. 30, 2023

Related U.S. Application Data
(63) Continuation of application No. PCT/US2021/043529, filed on Jul. 28, 2021.
(60) Provisional application No. 63/059,905, filed on Jul. 31, 2020.

(51) Int. Cl. G16H 40/67; G16H 20/17; G16H 10/60; A61M 5/42

(52) U.S. Cl. CPC G16H 40/67; G16H 20/17; G16H 10/60; A61M 5/427; G09B 5/02

(57) ABSTRACT

A method for tracking injection site information includes displaying, on a user interface, a plurality of injection zones, each of the plurality of injection zones representing a segment of the body suitable for medication injection and including a graphical indicator indicating a period of time since a most recent previous injection in the injection zone. The method also includes receiving, via the user interface, injection information relating to a new injection in a particular injection zone. The method also includes updating a graphical indicator of the particular injection zone on the user interface to indicate a new date and time of injection to the particular injection zone.

### RELATED U.S. APPLICATIONS

This application is a continuation patent application of PCT International Application No. PCT/US2021/043529, filed on Jul. 28, 2021, which claims priority to and the benefit of U.S. Provisional Application No. 63/059,905, filed on Jul. 31, 2020, each of which is hereby incorporated by reference in its entirety.

### BACKGROUND

### Field

Embodiments relate to systems and methods for managing illnesses and diseases, and, in particular, to systems and methods that provide smart, connected, end-to-end solutions for delivering personalized insights to patients or other users.

### Description

Diabetes is a group of diseases marked by high levels of blood glucose resulting from defects in insulin production, insulin action, or both. Diabetes can lead to serious complications and premature death. There are, however, well-known products and strategies available to patients with diabetes to help control the disease and lower the risk of complications.

Treatment options for diabetics include, for example, specialized diets, oral medications, and insulin therapy. A primary goal of diabetes treatment is to control a diabetic's blood glucose level in order to increase the chance of a complication-free life. Because of the nature of diabetes and its short-term and long-term complications, it is important that diabetics are constantly aware of the level of glucose in their blood and closely monitor their diet. For patients who take insulin therapy, it is important to administer insulin in a manner that maintains glucose levels, and accommodates the tendency of glucose concentration in the blood to fluctuate as a result of meals and other activities.

Healthcare professionals, such as doctors or certified diabetes educators (CDEs), offer counseling to diabetic patients regarding managing diet, exercise, lifestyle, and general health. When followed, this counseling can reduce complications associated with diabetes and allow diabetics to lead healthier and happier lives. Often, however, such counseling is only available by appointment, leaving diabetics without simple, quick, and readily available counseling regarding a healthy diabetic lifestyle.

### SUMMARY

For purposes of summarizing the described technology, certain objects and advantages of the described technology are described herein. Not all such objects or advantages may be achieved in any particular embodiment of the described technology. Thus, for example, those skilled in the art will recognize that the described technology may be embodied or carried out in a manner that achieves or optimizes one advantage or group of advantages as taught herein without necessarily achieving other objects or advantages as may be taught or suggested herein.

One embodiment is a method for tracking injection site information. The method includes displaying, on a user interface, a plurality of injection zones, each of the plurality of injection zones representing a segment of the body suitable for medication injection and including a graphical indicator indicating a period of time since a most recent previous injection in the injection zone. The method also includes receiving, via the user interface, injection information relating to a new injection in a particular injection zone. The method also includes updating a graphical indicator of the particular injection zone on the user interface to indicate a new date and time of injection to the particular injection zone.

Another embodiment is a system for tracking injection site information. The system includes an interactive user interface configured to display and receive user information and a memory having instructions that when run on a processor will perform a method including displaying, on the user interface, a plurality of injection zones, each of the plurality of injection zones representing a segment of the body suitable for medication injection and including a graphical indicator indicating a period of time since a most recent previous injection in the injection zone. The method also includes receiving, via the user interface, injection information relating to a new injection in a particular injection zone. The method also includes updating a graphical indicator of the particular injection zone on the user interface to indicate a new date and time of injection to the particular injection zone.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 is a block diagram illustrating an integrated disease management (IDM) system according to one embodiment.

FIG. 2 is a block diagram illustrating an embodiment of a learning management system for an integrated disease management system.

FIG. 3 is a flowchart illustrating an example process for updating content using the learning management system of FIG. 2.

FIG. 4 is a flowchart illustrating an example process for selecting and displaying content to a user based on a triggering event using the learning management system of FIG. 2.

FIG. 5 is a flowchart illustrating an example process for displaying content based on a scheduled event using the learning management system of FIG. 2.

FIG. 6 is a flowchart illustrating an example workflow process for structured education content.

FIG. 7 is a flowchart illustrating an example process for determining a patient goal or goals in an integrated disease management system.

FIG. 8 is a flowchart illustrating an example process for storing patient data in an integrated disease management system.

FIG. 9 is a flowchart illustrating an example process for displaying contextualized insights along with a graphical representation of patient data in an integrated disease management system.

FIG. 10 is an example screen capture of a user interface of the integrated disease management system according to one embodiment.

FIG. 11 is an example screen capture of the user interface illustrating a voice input function of the user interface.

FIG. 12 is an example screen capture of the user interface illustrating a text-based response to a user voice input according to one embodiment.

FIG. 13 is a flow chart illustrating an embodiment of a method for a voice input module of an integrated disease management system.

FIG. 14 is a flow chart illustrating an embodiment of another method for a voice input module of an integrated disease management system.

FIGS. 15 and 16 are example screen captures of home screens of a user interface of an integrated disease management system according to an embodiment.

FIGS. 17 and 18 are example screen captures of a learn module of a user interface of an integrated disease management system according to an embodiment.

FIGS. 19, 20, 21, and 22 are example screen captures of a goals module of a user interface of an integrated disease management system according to an embodiment.

FIGS. 23, 24, and 25 are example screen captures of a logging module of a user interface of an integrated disease management system according to an embodiment.

FIG. 26 is an example screen capture of a data module of a user interface of an integrated disease management system according to an embodiment.

FIGS. 27, 28, 29, 30, 31, and 32 are example screen captures of a goals module of a user interface of an integrated disease management system according to an embodiment.

FIGS. 33, 34, 35, 36, 37, 38, and 39 are example screen captures of a goals module of a user interface of an integrated disease management system according to an embodiment.

FIG. 40 is an example screen capture of a chatbot interface of a user interface of an integrated disease management system according to an embodiment.

FIGS. 41, 42, 43, 44, 45, 46, 47, 48, 49, 50, 51, and 52 are example screen captures of a logging module of a user interface an integrated disease management system according to an embodiment.

### DETAILED DESCRIPTION

### Introduction

Integrated disease management (IDM) systems and methods are described herein. As will be appreciated by one skilled in the art, there are numerous ways of carrying out the examples, improvements, and arrangements of the IDM systems and methods in accordance with embodiments of the present invention disclosed herein. Although reference will be made to the illustrative embodiments depicted in the drawings and the following descriptions, the embodiments disclosed herein are not meant to be exhaustive of the various alternative designs and embodiments that are encompassed by the disclosed invention, and those skilled in the art will readily appreciate that various modifications may be made, and various combinations can be made, without departing from the invention.

Although described herein primarily in the context of diabetes, the IDM systems or methods detailed below can be used to manage other types of diseases as well. These systems and methods can be used by many types of users, including, but not limited to, diabetic patients, non-diabetic persons, caregivers, and healthcare professionals or healthcare entities such as disease management companies, pharmacies, disease management-related product suppliers, insurers and other payers.

The IDM systems can be beneficial for all types of diabetic patients, including those with type 1 diabetes, type 2 diabetes, or a pre-diabetic condition. The IDM systems described herein can allow users to access readily available counseling information regarding a healthy diabetic lifestyle. The IDM systems can engage users in a manner that encourages them to maintain continuous (e.g., daily, weekly, or monthly) interaction with the IDM system to gain knowledge about diabetes and encourage them to lead an increasingly healthy lifestyle. Diabetes patients who engage with an IDM system such as described herein will often feel more in control of their diabetes management, which, in turn, to better patient outcomes. Often, the more a diabetic patient engages with the IDM system, the more satisfied they will feel with their life with diabetes (providing a desirable feeling of control). The IDM systems can use engagement, behavior design, and behavior change approaches to tailor the experience to each patient. The IDM system experiences can be designed to create more contextual, meaningful education that leads to more self-efficacy.

In an illustrative embodiment, the IDM systems include an interactive interface that is engaging, and that provides a way for users to seek information and support when needed so that they feel more in control of their condition. One or more features of the IDM systems can be based on behavioral science techniques that are designed to modify patient behavior.

In some embodiments, the IDM systems can use uploaded user health information to customize interactions with users. User health information can include data entered via the interactive interface, data uploaded from internet-enabled (“smart”) devices (such as smart insulin pens or pumps, diabetes monitors, fitness trackers, diet trackers, etc.), and other types of information. The IDM systems can analyze the uploaded health information to provide customized information to the user. The IDM system can be connected to additional outside services. For example, the IDM system can be connected to Apple® Healthkit®. Connecting the IDM system to outside services, such as Apple® Healthkit® and others, may further strengthen the IDM system's ability to tailor content for the user. For example, accessing Apple® Healthkit® may provide the IDM system additional information about the user. Additionally, the IDM system may provide information to the outside services connected to the system.

Example Devices that can Interface with the IDM Systems and Methods

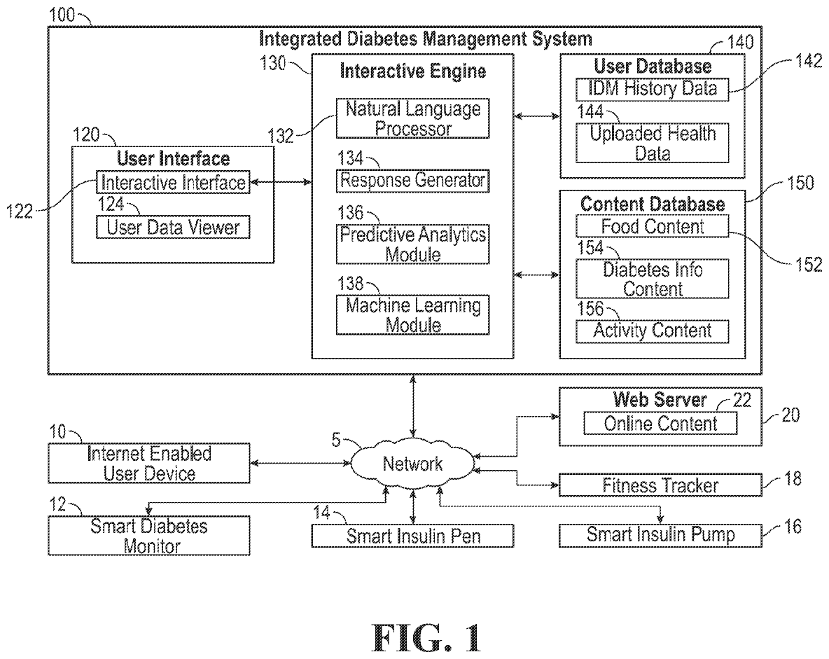

FIG. 1 is a block diagram that illustrates an integrated disease management (IDM) system 100 according to one embodiment in the context of diabetes management, as well as several additional devices that can communicate with the IDM system 100 over a network 5. In the illustrated embodiment of FIG. 1, these additional devices include an internet-enabled user device 10, a smart diabetes monitor 12, a smart insulin pen 14, a smart insulin pump 16, and a fitness tracker 18. These illustrated devices are provided by example only and other types of devices can also connect to the system 100 over the network 5. In some embodiments, one or more of these devices may be omitted and/or additional devices may be included.

The internet-enabled user device 10 can be any type of internet-enabled device without limit, including, a smartphone, tablet, laptop, computer, personal digital assistant (PDA), smartwatch, etc. In some instances, the internet-enabled user device 10 is a mobile device, such as any mobile device known in the art, including, but not limited to, a smartphone, a tablet computer, or any telecommunication device with computing ability, a mobile device connection module, and an adaptable user interface such as, but not limited to a touchscreen. A user typically possesses an internet-enabled user device 10, which can be used for various functions, such as sending and receiving phone calls, sending and receiving text messages, and/or browsing the internet.

The smart diabetes monitor 12 can be any type of internet-enabled diabetes monitor without limit. The smart diabetes monitor 12 can be configured to measure a user's blood glucose level, such as an electronic blood glucose meter or a continuous glucose monitor (CGM) system. The smart diabetes monitor 12 may be configured to upload information regarding a user's blood glucose level measurements to the IDM system 100. The measured blood glucose level and the time of measurement can be uploaded to the IDM system 100. In some embodiments, uploaded blood glucose level measurements are further associated with recently eaten foods and/or physical activity and this information can be uploaded to the IDM system 100 as well.

In some embodiments, a conventional, non-internet-enabled diabetes monitor can be used with the IDM system. Measurements from the conventional diabetes monitor can be entered or otherwise obtained via the internet-enabled user device 10 and uploaded to the IDM system 100 over the network 5.

The smart insulin pen 14 can be any internet-enabled device for self-injection of insulin without limit. Insulin pens typically provide the ability for a user to set and inject a dose of insulin. Accordingly, a user can determine how much insulin they need and set the appropriate dose, then use the pen device to deliver that dose. In an illustrative embodiment, a smart insulin pen 14 transmits information regarding the timing and dose of an insulin injection to the IDM system 100 over the network 5. In some embodiments, information about uploaded insulin injections is further associated with recently eaten foods or physical activity and this information can be uploaded to the IDM system 100 as well.

In some embodiments, a conventional, non-internet-enabled insulin pen can be used. Information about insulin injections from conventional insulin pens can be entered or otherwise obtained via the internet-enabled user device 10 and uploaded to the IDM system 100 over the network 5.

The smart insulin pump 16 can be any type of insulin pump including those that are internet-connected. The smart insulin pump 16 can be a traditional insulin pump, a patch pump, or any other type of insulin pump. The smart insulin pump 16 can upload information regarding the delivery of insulin to the patient to the IDM system 100 over the network 5. In some embodiments, the smart insulin pump 16 uploads information regarding the rate and quantity of insulin delivered by the pump.

In some embodiments, a conventional insulin pump can be used. Information about insulin delivery by the conventional insulin pump can be entered or otherwise obtained via the internet-enabled user device 10 and uploaded to the IDM system 100 over the network 5.

The fitness tracker 18 can be any device which measures (or otherwise obtains) health information (or other types of information) about the user. The fitness tracker 18 can be a device which measures patient vitals. In an illustrative embodiment, patient vital data includes, but is not limited to, heart rate, blood pressure, temperature, blood oxygen level, and/or blood glucose level. The patient vital data measurement values can be measured using sensors on the fitness tracker 18.

The information uploaded to the IDM system 100 by the internet-enabled device 10, the smart diabetes monitor 12, the smart insulin pen 14, the smart insulin pump 16, and/or the fitness tracker 18 or one or more additional devices can be associated with a particular user. The information can be used to customize interaction between the user and the IDM system 100, for example, allowing the IDM system 100 to provide better answers or recommendations for the user. In some embodiments, the IDM system 100 analyzes the uploaded information to evaluate the health of the user.

Also shown in FIG. 1 is a web server 20. The web server may provide online content 22, which can be referred to, referenced by, or otherwise used by the IDM system 100. In an illustrative embodiment, the web server 20 provides a website accessible by users over the network 5. The website can include online content 22 related to diabetes, food choices, exercise, or other topics. As will be described below, the IDM system 100 can link users to the web server 20 to access the online content 22 in response to user questions.

The network 5 can include any type of communication network without limit, including the internet and/or one or more private networks, as well as wired and/or wireless networks.

### Example IDM Systems and Methods

The IDM system 100 will now be described with reference to the embodiment illustrated in FIG. 1. The IDM system 100 may be embodied in a single device (e.g., a single computer or server) or distributed across a plurality of devices (e.g., a plurality of computers or servers). The modules or elements of the IDM system 100 can be embodied in hardware, software, or a combination thereof. The modules or elements may comprise instructions stored in one or more memories and executed by one or more processors.

Each memory can be a RAM memory, a flash memory, a ROM memory, an EPROM memory, an EEPROM memory, a register, a hard disk, a removable disk, a CD-ROM, or any other form of storage medium known in the art. Each of the processors may be a central processing unit (CPU) or other type of hardware processor, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or in the alternative, the processor may be any conventional processor, controller, microcontroller, or state machine. A processor may also be implemented as a combination of computing devices, for example, a combination of a DSP and a microprocessor, a plurality of microprocessors, one or more microprocessors in conjunction with a DSP core, or any other such configuration. Exemplary memories are coupled to the processors such that the processors can read information from and write information to the memories. In some embodiments, the memories may be integral to the processors. The memories can store an operating system that provides computer program instructions for use by the processors or other elements included in the system in the general administration and operation of the IDM system 100.

In the illustrative embodiment shown in FIG. 1, the IDM system 100 includes a user interface 120, an interactive engine 130, a user database 140, and a content database 150. In some embodiments, one or more of these elements can be omitted. In some embodiments, the IDM system 100 contains additional elements.

The user database 140 can comprise a single database or a plurality of databases. In an exemplary embodiment, users of the IDM system 100 each have an account with the IDM system 100. Information regarding user accounts can be stored in the user database 140. The user database 140 can also store additional information associated with the user account. For example, the user database 140 can store IDM history data 142 and uploaded health data 144.

In an illustrative embodiment, IDM history data 142 is data generated and stored during a user's previous interactions with the IDM system 100. This can include previous inquiries submitted by the user; previous responses provided by the user; user-entered preferences; and/or a log indicating the timing of the user's interactions with the IDM system 100, among other things. The IDM system 100 can automatically add IDM history data 142 as the user continues to use and/or interact with the IDM system 100. The IDM history data 142 can be used by a predictive analytics module 136 and a machine learning module 138 of the interactive engine 130 (or other modules of the IDM system 100) to customize future interactions between the IDM system 100 and the user. As a user interacts with the IDM system 100, the IDM history data 142 associated with the user's account in the user database 140 grows, allowing the IDM system 100 to know the user better, provide better content, and create a more engaging experience. In some embodiments, this increases the efficacy of the IDM system 100.

The user database 140 also stores uploaded health data 144 associated with a user's account. The uploaded health data 144 can include the information entered by a user on the internet-enabled user device 10 or uploaded by the smart diabetes monitor 12, smart insulin pen 14, smart insulin pump 16, and/or fitness tracker 18 (described above). The uploaded health data 144 can also include additional information produced by the IDM system 100 upon analysis of the user's uploaded data. For example, upon analysis of the user's uploaded data, the IDM system may generate health trend information, which can also be stored among the uploaded health data 144 associated with the user's account in the user database 140. In some embodiments, uploaded health data 144 can include information uploaded or entered by a healthcare provider, such as a doctor, nurse or caregiver. Data that is gathered or measured by connected devices and stored in the user database 140 may include measured patient disease management data. Data that is entered by the user into the user database 140 may include user-derived patient disease management data.

In the illustrative embodiment, the IDM system 100 also includes a content database 150. The content database 150 can be a single database or a plurality of databases. The content database 150 includes content that is delivered to users during user interaction with the IDM system 100. The content can include diabetes education information. In some instances, the content is developed, selected, and/or curated by healthcare professionals, such as doctors or CDEs. The content can be similar to that which is provided by healthcare professionals during in-person counseling sessions. However, content on the IDM system 100 is available to the user at any time and accessible, for example, on the internet-enabled device 10.

In the illustrated embodiment, the content database 150 includes food content 152, diabetes information content 154, and activity content 156. In an illustrative embodiment, food content 152 can be developed and curated to encourage users to eat healthy, while still allowing them to eat foods that they enjoy.

Diabetes information content 154 can be developed and curated to provide answers to common questions asked by diabetic patients. Other types of diabetes information content 154 can also be included, such as protocols for managing diabetes or other diseases.

Activity content 156 can be developed and curated to provide information about healthy lifestyle choices and physical activities for diabetics. The activity content 156 can be developed by healthcare professionals.

Food content 152, diabetes information content 154, and activity content 156 are shown by way of example of certain types of content only, and other types of content can be included in addition to or in place of one or more of the illustrated types of content.

The IDM system 100 can include a user interface 120 and an interactive engine 130. The user interface 120 can provide an interface by which the IDM system 100 interacts with or displays information to users. The user interface 120 can be accessible to the user over the network 5. For example, a user can access the user interface 120 on the internet-enabled user device 10. The user interface 120 can include an interactive interface 122 and a user data viewer 124. In some embodiments, the interactive interface 122 is an interactive application, such as a smartphone, tablet, or computer application. In some embodiments, the interactive interface 122 is an interactive website. In a non-limiting example, the interactive interface 122 is a chatbot.

The interactive interface 122 relays inputs and outputs between a user and the interactive engine 130. The interactive engine 130 processes inputs and outputs to provide an interactive experience for the user. The interactive engine 130 also retrieves information from the user database 140 and the content database 150. For example, in interacting with a user, the interactive engine 130 may access the user database 140 to obtain the user's IDM history data 142 and uploaded health data 144. In an illustrative embodiment, the interaction with the user is customized based on the user's IDM history data 142 and uploaded health data 144. Similarly, the interactive engine 130 can retrieve content from the content database 150. The interactive engine 130 can retrieve content from the content database 150 based on user inputs (e.g., questions, responses, and selections), as well as user information stored in the user database 140. Through the interactive interface 122, the interactive engine 130 provides engaging and informative interactions with the user that allows the user to feel in control of his or her diabetes management and gain diabetes education.

The interactive engine 130 can include a natural language processor 132, a response generator 134, a predictive analytics module 136, and a machine learning module 138. In some embodiments, one or more of these elements can be omitted or combined with another element. In some embodiments, the interactive engine 130 contains additional elements.

The natural language processor 132 and the response generator 134 can allow the interactive interface 130 to provide a simple interaction experience via the interactive interface 122. For example, in an illustrative embodiment, the natural language processor 132 and the response generator 134 allow a user to have an interactive chat (written or spoken) with the IDM system 100.

The natural language processor 132 can parse user inputs into a machine-understandable format. For example, in an illustrative embodiment, the interactive interface 122 allows a user to enter a natural language question. The natural language processor 132 can parse the question such that it can be understood by the interactive engine 130. As another embodiment, the interactive interface 122 can allow the user to speak a question. The natural language processor 132 can include a voice recognition module that can recognize the spoken question and parse the question such that it can be understood by the interactive engine 130.

The response generator 134 formulates responses to user inputs. The response generator 134 can receive information from the natural language processor 132. In an illustrative embodiment, responses generated by the response generator 134 include an answer to the user's question. Alternatively, the responses can include requests for additional information from the user. The request for additional information can be provided as a question prompt or one or more options from which the user can select. The response generated by the response generator 140 can be stylized in the “personality” of the IDM system 100 as mentioned above.

The interactive engine 130 can also include a predictive analytics module 136 and a machine learning module 138. In an illustrative embodiment, the predictive analytics module 136 uses information in the user database 140 (such as IDM history data 142 and uploaded health data 144) to predict content that a user will enjoy or that will be beneficial to the user. For example, based on uploaded health data 144, the predictive analytics module 136 can select content to present to the user designed to help the user manage his or her blood sugar.

In an illustrative embodiment, the machine learning module 138 analyzes information in the user database 140 (such as IDM history data 142 and uploaded health data 144) to provide inputs which can be communicated to the predictive analytics module 126. For example, the machine learning module 138 can learn about a user based on past interactions with the IDM system 100 and generate data which is used by the predictive analytics module 136 to customize content for future interactions. Thus, the more a user interacts with the IDM system 100, the more personalized interaction with the system will become. In some instances, personalized interaction increases the efficacy of the IDM system 100.

The user interface 120 can also include a user data viewer 124. The user data viewer 124 can be a portal that allows a user to access information related to their account.

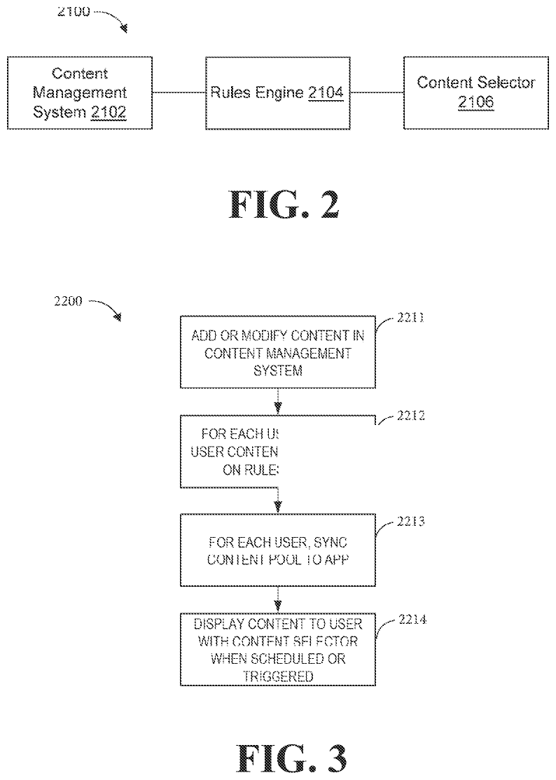

FIG. 2 is a block diagram illustrating an embodiment of a learning management system (LMS) 2100 that is configured to deliver personalized content to a user based on an evolving user profile. The LMS 2100 can be implemented by the IDM 100 described above. For example, the LMS 2100 can be implemented by the interactive engine 130 described above. In the illustrated embodiment, the LMS 2100 includes a content management system 2102, a rules engine 2104, and a content selector 2106.

In some embodiments, the LMS 2100 is driven, at least in part, by rules and user profiling. Over time, the LMS 2100 builds a user profile for each user. The user profile can be based on initial onboarding questions (e.g., questions asked of the user at the time of initial account creation) as well as additional information learned about the user as the user continues to interact with the LMS 2100. In some embodiments, rules applied by the LMS 2100 can be either explicit or non-explicit (i.e., “fuzzy”). Non-explicit or fuzzy rules can be based on a distance algorithm that determines a distance value between different types of content and returns content that is within a threshold range. For example, as will be described in more detail below, content in the LMS 2100 can be labeled with one or more tags. Relations between the tags can be used to determine distances between the content that can be used by the non-explicit of fuzzy rules of the LMS 2100.

Interactions between the LMS 2100 and the user (e.g., dialog and testing) can be dynamic based on user selections and answers. As the user provides additional information to the LMS 2100, the LMS 2100 adds this information to a dynamic user profile. Thus, the LMS 2100 can be said to involve continuous profiling of the users. As the profile for each user continues to evolve, this leads to new workflows and content that will be made available to the user in a customized and tailored way.

In the LMS 2100, the content management system (CMS) 2102 can store the universe of content items available for all users. The CMS 2102 can be a database or other method of storing the content. Various types of content items are available, including tutorials, videos, recipes, activities, tips, announcements, insights, follow-ups, praise, quizzes, patient health goals, etc. In some embodiments, the content items in the CMS 2102 are provided and/or curated by health care professionals or CDEs.

Each content item in the CMS 2102 can be labeled with one or more tags. The tags can be initially assigned when content is created and added to CMS 2102. In some embodiments, tags can be added, modified, or reassigned over time. The tags can be used for labeling and organizing content items within the CMD 2102. The tags can also be used for content selection (e.g., deciding which content to make available to which users) as described below.

Example tags can include “activity_less,” “activity_daily,” “activity_more,” “activity_no,” “gender_male,” “gender_female,” “gender_noanswer,” among many others. These tags can be used to identify content items that may be relevant to users that have profiles that relate to the tags. For example, a user's profile may indicate that they are generally active on a daily basis. As such, content items associated with the “activity_daily” tag may be deemed to be relevant to the particular user.

As mentioned above, onboarding questions may be initially used to identify which tags are relevant for a user. Then, as the users profile dynamically grows over time, the LMS 2100 may use the additionally learned information to change the group of tags that may be relevant for a user. In this way, users can be dynamically associated with changing groups of tags to provide an individualized content pool that is tailored to their particular profile.

In some embodiments, tags can be related to other tags. For example, a tag can be associated with an affinity tag. An affinity tag can be a tag related to the initial tag that may also be selected when the initial tag is selected. For example, a recipe can be tagged specifically with a tag indicative of a type of food. For example, a quiche recipe can be tagged with “quiche.” “Eggs” may be an affinity tag associated with the tag “quiche.” Affinity tags can be used to identify content items that are not specifically related to the initial tag. For example, the LMS 2100 can identify that the user is interested in a quiche recipe, and then can follow up with additional information about other eggs recipes using the affinity tag. This may allow the LMS 2100 to continue to develop the user's profile in other ways that are not directly related to the initial tag “quiche.”

In some embodiments, tags can also be associated with anti-affinity tags. Anti-affinity tags can be the opposite of affinity tags. For example, these can be tags that are cannot be selected with another tag. As one example, the user's profile may indicate that they are currently using a non-injection based therapy for treating their diabetes. Anti-affinity tags can be used to ensure that injection-based content (which is irrelevant to this particular user) is not provided.

Content items can be tagged with one or more tags. For example, a content item can be associated, with one, two, three, four, five, six, or more content tags. Tags themselves can be associated with other tags using affinity and anti-affinity tags as described above.

In some embodiments, content items can be organized into clusters. For example, based on the tags, each content item can be part of a cluster. Each cluster can use distance rules to determine the distance to every other cluster in the CMS 2102. Content recommendations can begin with the user's closest cluster and head outward in a simple fashion. For example, after recommending content items in the user's closest cluster, the LMS 2100 can move to the next closest cluster, and so on. This can ensure that the content is presented to the user beginning with the most relevant content, and then branching outward to continue to develop the user's profile.

There are several ways that distances can be calculated between content items or between data clusters. For example, content items with matching tags can be determined to have a distance of 0 between them. Content items with affinity tag matches can be determined to have a distance of 1 between them. For example, tags A and B can be determined to be affinity tags. Thus, a content item tagged with A and a content item tagged with B can be determined to have a distance of 1 between them. Content items with anti-affinity tag matches can be determined to have a distance of 1000 between them. For example, tags A and C can be determined to be anti-affinity tags. Thus, a content item tagged with A and a content item tagged with C can be determined to have a distance of the 1000 between them. Content items that include tags that are associated with matching affinity tags can be determined to have a distance of 10 between them. For example, tag A can be an affinity tag of D, and tag D can be an affinity tag of E. Thus, a content item tagged with A and a content item tagged with E can be determined to have a distance of 10 between them. As the relationships between affinity tags becomes more distant, the determined distance between tags can increase. For example, assume A and G are affinity tags, I and K are affinity tags, and G and K are affinity tags. A and I are distantly related through several affinity tag connections. Thus, a distance between content tagged with A and content tagged with I can be 25, for example. In some embodiments, content tagged with wholly unrelated tags can be determined to have a distance of 50. In some embodiments, distance is determined by taking the average for all pairwise distances between any two items and that is the distance between the two items. In some embodiments, if the tags are an exact match between two items taking a pairwise comparison is not necessary and the distance is determined to be 0. The distance calculation methods described in this paragraph are provided by way of example only, and other methods for determining distances between tagged content items are possible.

The rules engine 2104 may be configured to maintain a personalized content pool for each individual user. The content pool comprises a subset of content items from the CMS 2102 that are available for display to a particular user. Items in the user's content pool are chosen based on rules, tags, and user's profile. Thus, while the CMS 2102 includes the universe of content which can be available to all users, the rules engine 2104 selects particular content from the CMS 2102 for each individual user based on the user's profile and the content tags. As described below, the content can include patient goals, and the rules engine 2104 can determine particular goals from the CMS 2102 for the user.

In some embodiments, the rules can be scheduled rules or triggered rules. Scheduled rules can be rules that are scheduled to run at a particular time. For example, a scheduled rule may be: do X every Sunday at 6:15 PM, or do Y every data at 7 AM. In contrast with scheduled rules, triggered rules are configured to occur do to a particular event occurring for the user. For example, a triggered rule may be: when X occurs, do Y. Triggered rules can be triggered by many different types of events. For example, triggers can include: BGM events; fasting BGM Events; pre-prandial BGM event; post-prandial BGM events; insulin events; basal insulin events; bolus insulin events; study start events; next appointment events; meal events; step events; mood events; communication events; chat message sent events; chat message received events; content updated events; profile updated events; content viewed events; content expired events; launch events; etc.

Rules can also include an indication of how content items can be sent/displayed to the user. For example, some rules can specify that a content item should be immediately sent or displayed to the user. Content can be sent to the user the text (SMS), push notification, email, or other communication methods. Other rules can specify that the content item should be added to the content pool for possible display to the user later. For example, a rule can indicate that 15 new recipes should be added to the user's content pool. As will be discussed below, the content selector 2104 can be used to select and display individual content items from the user's content pool to the user.

Some rules can identify a particular item of content. For example, a rule may specify a particular ID of a content item. This would be an example of an explicit rule. In other cases, a rule may not explicitly identify a particular item of content. For example, a rule may specify a content type generally (e.g., recipes) and then may provide content based on a distance-matching algorithm as described above. This would be an example of non-explicit or fuzzy rule. In this case, content is selected for the user based on the user's profile and the distance-matching algorithm.

In some embodiments, rules can include a specified priority. For example, the rules engine 2104 may buffer incoming changes for a short period of time (e.g., seconds), and multiple rules can fire based on the same trigger. Thus, for each content type, only one rule may be allowed to generate output for each firing run (per user). To control which rule takes priority in the case, rules can include priorities, and rules with higher priorities will trump rules with lower priorities. Priority values can be specified in a number of ways. For example, priority values can range from 1 to 2100, or general priority categories (e.g., Low, Medium, High) can be used.

Similarly, certain rules can be set to supersede other rules. For example, a supersedes indicator followed by a rule identifier can express the concept that one rule will always take precedence over another (and remove existing content from the pool from the superseded rule). Rules can include additional limits on how often a rule can be executed. Some limits can be set on a per day, per week, per month, or per user basis. In some embodiments, rules can further include additional conditions that must be met for the rule to be executed. For example, rules can be configured with when clauses that cause the rule to be executed only when specified user state conditions are met. For example, a rule can include a when clause that causes the rule to only be executed when the BGM measurement is within a normal range. Other examples can include: when last 1 BGM>200; when last 3 BGM>280; when BGM count <1 in last 5 days; when insulin count >3 in last 12 hours; and many others. In some embodiments, rules can include optional active or activation clauses. Activation clauses can put temporal boundaries on rules. These may be useful when have patient appointments or want to schedule something relative to another date. Finally, rules can also optionally include an expiration term. This can limit how long a particular content item remains in the user's content pool.

Several example rules that can be executed by the rule engine 2104 will now be described. These rules are provided by way of non-limiting example, and many other types of rules are possible.

In a first example, a rule may state:

Rule Announcement

Triggered By Content Update

Add up to 5 Announcement

Do Not Reuse

Priority 2100

This rule queues up to 5 announcements that haven't been seen by the user with highest priority. ‘Do Not Reuse’ indicates that the rule engine 2104 not re-add previously viewed content for a user. In some embodiments, if not specified, the default is to reuse content. When executed the rule will query for all announcements sorted by newest, and add up to five to the user's pool.

As another example, a rule may state:

Rule InitRecipes

Triggered By Launch

Add up to 15 recipe

With Max Distance 200

This rule may be executed each time the user launches or change their profile and is configured to add recipes to the queue up to 15 total recipes (not 15 new recipes). The term “With Max Distance” specifies how ‘different’ content can be and still be added to the User's Pool. The higher the value, the less appropriate content can be. This allows implementations of non-explicit or fuzzy rules as mentioned above.

As another two rules may state:

Rule ONEBGHIGH

Triggered By BGM

Add insights

When Last BGM>200

ContentId: Z3WbRWKjkcAkwAWMMq42O

Priority 95

Limit 1 per 7 days

Expire in 24 hours

Rule THREEBGHIGH

Triggered By BGM

Add insights

When Last 3 BGM>200

ContentId: Z3WbRWKjkcAkwAWMMq42O

Priority 95

Supersedes ONEBGHIGH

Limit 1 per 7 days

Expire in 24 hours

These rules add specific content items when triggered by certain BGM measurements. Thus, these rules queue the BGM high insight max once a week on high BG measurement. Rule THREEBGHIGH supersedes Rule ONEBGHIGH because it includes “Supersedes ONEBGHIGH.” Thus, ONEBGHIGH cannot be executed if THREEBGHIGH is already queued.

As another example, a rule may state:

Rule FollowUpRecipe

Queue FollowUp

Triggered By recipe Viewed

Expire in 15 days

Priority 97

This rule queues a follow up after a recipe has been viewed. This may allow the LMS 2100 to continue to develop the user's profile by requesting additional information about whether a user liked a recipe after trying the recipe. This additional information can be used to tailor additional content to the user in the future. These rules may be stored in a memory of the system as executable instructions and then executed by a processor that is configured to run the rules from executable instructions.

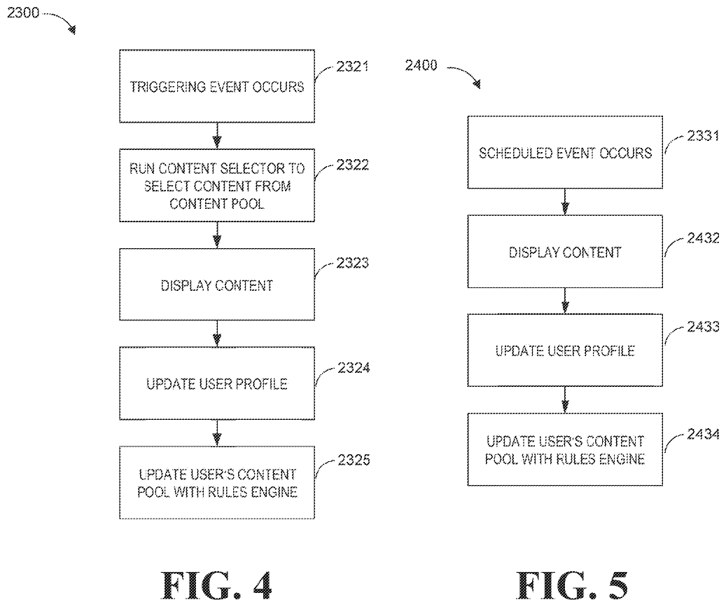

As shown in FIG. 2, the LMS 2100 also includes a content selector 2106. The content selector 2106 determines which content from the content pool to display to the user. Selections can be made based on triggering/reactive events (described with reference to FIG. 4) or scheduled events (described with reference to FIG. 5). Thus, the content selector 2106 determines when and how to display individual content items from the content pool to the user. In the case of patient goals, the content selector 2106 can identify a particular subset of patient goals for display to the user. Additional examples of triggers and non-limiting examples of corresponding reactive events are provided in Table 1.

TABLE 1

| Example Trigger | Example Reactive Event |
|---|---|
| Number of steps has increased by 1,000 | Conversation with direction to |
| steps over 3 consecutive days | personalized article content |
| Number of steps has decreased by 500 | Conversation with recommendation for |
| steps over 3 consecutive days | personalized goal setting |
| Connected to application such as Apple | Conversation with recommendation for |
| Health | personalized goal setting |
| Took 5,000 steps 3 times in a 7-day period | Congratulatory message with |
| | recommendation for personalized goal |
| | setting |
| BG <70 mg/dl logged and an increase of | Conversation with direction to |
| 500 steps | personalized article content |
| Increased activity, BG in desired range | Conversation with direction to |
| | personalized article content |
| Decreased activity, increased blood | Conversation with direction to |
| glucose | personalized article content |
| BG high, corresponding insulin not logged | Conversation with direction to |
| | personalized article content |
| BG & insulin both logged 3 days in a row | Congratulatory message with |
| | recommendation for personalized goal |
| | setting |
| Restless sleep, BG >180 mg/dl logged | Conversation with direction to |
| | personalized article content |
| 2 nights < 2 waking with BG in range after | Congratulatory message with direction to |
| restless sleep, >180 mg/dl | personalized article content |
| 2 consecutive breakfast blood glucose | Conversation with direction to |
| readings >180 mg/dl | personalized article content |
| 2 non-moming BG readings >180 mg/dl | Conversation with direction to |
| | personalized article content |
| 2 consecutive dinner BG readings >180 | Conversation with direction to |
| mg/dl | personalized article content |
| 3 consecutive bedtime BG readings >180 | Conversation with direction to |
| mg/dl | personalized article content |
| BG <70 mg/dl | Conversation with direction to |
| | personalized article content |
| BG <70 mg/dl, twice in a 7 day period | Conversation with direction to |
| | personalized article content |
| BG <80 mg/dl in the morning | Conversation with direction to |
| | personalized article content |
| BG <80 mg/dl at lunch time | Conversation with direction to |
| | personalized article content |
| BG <80 mg/dl at dinner time | Conversation with direction to |
| | personalized article content |
| BG <80 mg/dl at bedtime | Conversation with direction to |
| | personalized article content |
| Blood glucose readings two days in a row | Conversation with direction to |
| are between 80-180 mg/dl, following one | personalized article content |
| to two readings over 180 mg/dl | |
| Did not log blood glucose at lunch, after | Conversation with direction to |
| logging last 3 days | personalized article content |
| Did not log blood glucose that day, after | Conversation with direction to |
| logging last 2 days | personalized article content |
| Did not log blood glucose at bedtime, after | Conversation with direction to |
| logging last 3 days | personalized article content |
| BG logged 3 days in a row | Congratulatory message with |
| | recommendation for personalized goal |
| | setting |
| Connected to blood glucose meter, such as | Congratulatory message with direction to |
| Accu-Chek meter | personalized article content and/or |
| | instructions related to use of meter with |
| | App |
| Blood glucose readings three days in a row | Conversation with direction to |
| include ones that are lower than 80 or | personalized article content |
| higher than 180 mg/dl | |
| Second BG >180 mg/dl | Conversation with direction to |
| | personalized article content and/or |
| | instructions |
| BG >180 mg/dl more than one time in 7 | Conversation with direction to |
| days | personalized article content |
| BG >180 mg/dl 3x in 7 days | Conversation with direction to |
| | personalized article content |
| Asked 3+ questions over 3 consecutive | Conversation with direction to |
| days | personalized article content |
| Read 3+ articles over 3 consecutive days | Conversation with direction to |
| | personalized article content |
| Open the app and engaged (chat, read, log) | Congratulatory and request for review |
| at least once consecutively over 5 days | |
| Read article twice (example: “Fast Food | Conversation with question to ask user if |
| Options for People with Diabetes“article”) | they'd like to read another article (example: |
| | “10 Tips for Healthier Fast Food Choices”) |
| Read a recipe | Conversation with direction to more recipe |
| | or food content x hours after trigger |
| Insulin dose was not logged in that day, | Conversation with direction to |
| after logging for the last 2 days | personalized article content |
| Insulin dose was not logged in for lunch, | Conversation with direction to |
| after logging for the last 2 days | personalized article content |
| Insulin dose was not logged in for, bedtime | Conversation with direction to |
| after logging for the last 2 days | personalized article content |
| Insulin logged 3 days in a row | Congratulatory message with |
| | recommendation for personalized goal |
| | setting |
| Change in dose leads to low BG | Conversation with instruction to consult |
| | care provider |
| Change in insulin brand | Conversation with question to user asking |
| | if they meant to select a change in brand; |
| | continued conversation with questions to |
| | understand why there was change |
| Addition of bolus insulin | Conversation to confirm addition of bolus |
| | insulin, and, following receipt of |
| | confirmation, direction to article, such as |
| | “What is Basal-Bolus Insulin Therapy” |
| | article |
| 1st time logging insulin | Conversation with question to user asking |
| | if they're new to insulin, and if so, direction |
| | to article, such as “Starting Insulin? Here's |
| | What You Need to Know” article |
| User exported data report | Conversation with questions to user |
| | regarding identity of recipient of report |
| | and/or content of report 24 hours after |
| | trigger |
| Waking up 2 or more times during the night | Conversation with direction to |
| | personalized article content |
| Blood glucose readings two days in a row | Conversation with direction to |
| are between 80-180 mg/dl, following one | personalized article content |
| to two readings over 180 mg/dl | |
| Insulin never logged | Conversation with direction to logging |
| | module |
| No insulin logged in more than 1 week | Conversation with direction to logging |
| | module |
| Insulin logged but injection site not tracked | Conversation with direction to article |
| | regarding site rotation |
| Insulin logged and injection site tracked | Congratulatory message and conversation |
| twice | with direction to article about good site |
| | rotation technique |
| Insulin logged and injection site tracked | Congratulatory message and conversation |
| twice in one week. | with direction to article about good site |
| | rotation technique |

The relationships between the example triggers and example reactive events in Table 1 are for illustrative purposes only. It is contemplated that the example triggers of Table 1 may be associated with reactive events different from, or in addition to, the example reactive events listed in Table 1. The “conversations” and/or “messages” described in the example reactive events may be performed using any content display or communication method described herein. For example, the “conversations” and/or “messages” can be displayed in the app or provided via text message, email, or some other communication method. As described above, in some embodiments, rules such as those described in Table 1 can include a specified priority. Certain rules can supersede other rules. In some embodiments, certain rules may be designed to repeat automatically or to repeat after a certain period of time. Certain rules may be designed to repeat a finite number of times or to occur only once. In some embodiments, certain rules can expire after a predetermined period of time after the rules are triggered, for example, 24 hours of 10 days. In some embodiments, certain rules can expire in response to an action by the user, for example selecting an option in the app or completing the reactive event. In some embodiments, a notification or reactive event can be displayed or otherwise active until expiration of the rule, for example, due to the passage of a predetermined period of time and/or due to an action by the user.

As described above, interactions (e.g., dialog and testing) between the LMS 2100 and the user can be dynamic based on user selections and answers. For example, in Table 1, the reactive event for the trigger “BG<70 mg/dl” is “Conversation with direction to personalized article content.” The conversation with the user, for example using a chatbot interface, can result in the IDM providing a recommendation for an article about exercise, a recommendation for a recipe, or an option of either an article about exercise or a recipe, depending on the user's selections, answers, and/or user profile.

In some embodiments, rules may be assigned to a particular user based on a number of factors including region, diabetes type, treatment type, or other information in the user's profile. In some embodiments, certain rules can be activated or deactivated by the user.

In some embodiments, a trigger can be activated when a user scans a machine-identifiable code such as a barcode or QR code using a device connected to the IDM, such a camera, optical scanner, or barcode reader. In some embodiments, the user device 10 can include a camera configured to capture and read a machine-identifiable code. In some embodiments, scanning of a machine-identifiable code, for example on a website, product, or packaging, can initiate a reactive event in which new content is shown to or made available to the user, the user is navigated to a different part of the IDM, or a different chat dialogue is presented to the user. For example, scanning a code on an insulin pen, such as the BD Nano PRO™ from Becton Dickinson, or the packaging of the insulin pen can make content related to the insulin pen, such as instructions for use or educational content related to insulin delivery, available to the user. As another example, scanning a machine-identifiable code on a package for pen needles, such as a BD pen needle box, can provide access to educational content related to injection technique, such as the BD and Mc™ interface from Becton Dickinson. In some embodiments, the IDM may store such content in a memory prior to scanning of the machine-identifiable code, but restrict the user from accessing the content until the machine-identifiable code is scanned.

FIG. 3 is a flowchart illustrating an example process or method 2200 for updating content in an individual user's content pool using the learning management system 2100. The method 2200 can begin at block 2211 at which content in the CMS 2102 is added or modified. Updating or modifying content in the CMS 2102 can trigger the LMS 2100 to update the content pool for each user so that the new or modified content can be disseminated to the users.

The method 2200 can move to block 2212 at which, for each user, the content pool is updated using with rules engine 2104. At this step, the rules are applied for each user, taking into consideration each user's dynamically customized profile. This selects contents items from the CMS 2102 and adds them to each user's content pool. In some embodiments, the content pool for each user is customized or tailored specifically for them based on the user's dynamically customized profile, the tags associated with the content items, and the distance algorithm described above.

Next, the method 2200 can move to block 2213, at which, for each user, the user's content pool is synced to the application. For example, the content can be downloaded (or otherwise linked) onto the user's mobile device. In some instances, the content is not yet displayed to the user. Rather, at block 2213, the content pool is merely made available for future display to the user.

Finally, at block 2214, the content selector 2106 selects and displays content to the user when scheduled or triggered. That is, from among the content items in the content pool, the content selector 2104 chooses and displays content information to the user.

FIG. 4 is a flowchart illustrating an example process 2300 for selecting and displaying one or more content items to a user based on a triggering event using the learning management system 2100. The method 2321 may begin at block 2321 when a triggering event occurs. Several example of triggering events have been described above. As one example, the user may send a message using the system requesting a pizza recipe. At block 2322, the content selector 2322 is executed to select a content item from the content pool. Continuing with the pizza recipe example, the content selector may determine if the content pool contains a pizza recipe. Because the content pool has been previously updated and customized for the specific user, the likelihood that a pizza recipe that the user will like is increased. If the content pool does not include a pizza recipe, the content selector may return the most relevant content based on the content tags and the distance-matching algorithm.

At block 2323, the returned content item is displayed to the user. For example, the content item can be displayed in the app or provided via text message, email, or some other communication method. At block 2324, information about the displayed content is used to update the user's profile. The content may be removed from the user's pool as already having been displayed. One or more follow-ups with the user regarding the content may be set. At block 2325, the updated user's profile is used to update the user's content pool with the rules engine 2325. That is, based on this interaction, the content pool available to the user for future interactions may be dynamically adjusted.

FIG. 5 is a flowchart illustrating an example process or method 2400 for displaying content based on a scheduled event using the learning management system 2100. In this example, a scheduled event occurs at block 2431. Content associated with the scheduled event is displayed to the user at block 2432. Then, similar to the method 2300, the user's profile can be updated (block 2433) and the user's content pool can be updated (block 2434) based on the interaction.

In some embodiments, the LMS 2100 described above can be used to provide structured education content and workflows to users. The LMS 2100 may guide the user through the content in manner designed to facilitate understanding and learning. In this example, the structured education content is focused on injection therapy. The content can be tagged in the CMS 2102 with an “injection therapy” tag. Further, the IDM can personalize the content to the user's emotional and functional need. For example, the content can be dynamic to the particular patient's type of injection therapy. This can ensure the patient's comfort and understanding of the subject and support the patient at home as if they were sitting with a CDE or other healthcare professional.

In some embodiments, content can be divided into different topics, with different subjects available under each topic. Again, content tags can be used to identify topics and subjects. In some embodiments, the content can be delivered to the user as text or video tutorials. After completing a topic plan, the user's comfort level can be assessed. If the user is comfortable with the material, the LMS will advance to additional material. If not, the content is offered again. In some embodiments, upon completion of the topic, the user receives a summary of the subject matter.

In the context of injection therapy, example topic plans can include overcoming mental hurdles, an introduction to injection mechanics, how to injection (segmented for syringe and pen users), injection best practices, learning how to deal with hypos/hypers, advanced injection therapy, understanding diabetes, and blood glucose tracking and best practices.

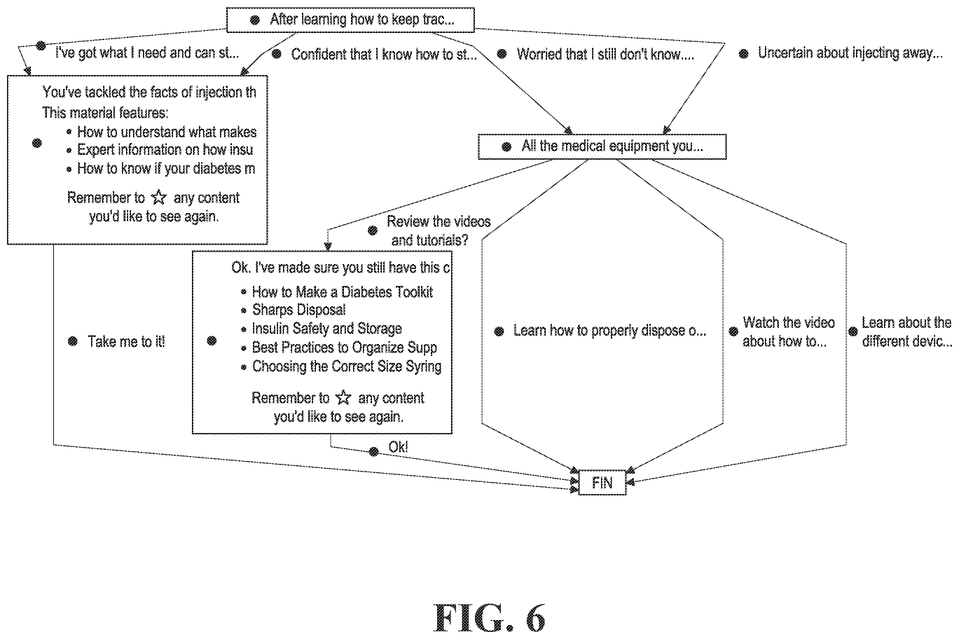

FIG. 6 is a flowchart illustrating an example workflow process for structured education content. Rules in the LMS 2100 may guide the user through the workflow process to ensure comfort and mastery of the material. As shown in FIG. 6, the workflow begins after the user has been provided an initial tutorial or information on learning how to keep track of injections. The user is given selectable options to assess his or her comfort level. For example, in the illustrated embodiment, the options include, “I've got what I need and can start,” “Confident that I know how to start,” “Worried that I still don't know,” and “uncertain about injecting any way.” Depending on the user's selection, the user is directed to additional content or to review the previous content to gain confidence and mastery. As the user progresses through the workflow, the user's profile can be continually and dynamically updated to provide additional customization and tailored content for future interactions.

In some embodiments, an IDM, such as the IDM 100 of FIG. 1, can include a voice input module, which can for example, be part of the user interface 120. The voice input module may be configured to allow a user to input data into the system by speaking. An example screen 3200B of an interactive interface that includes a voice input module is shown in FIG. 11, which is described in more detail below.

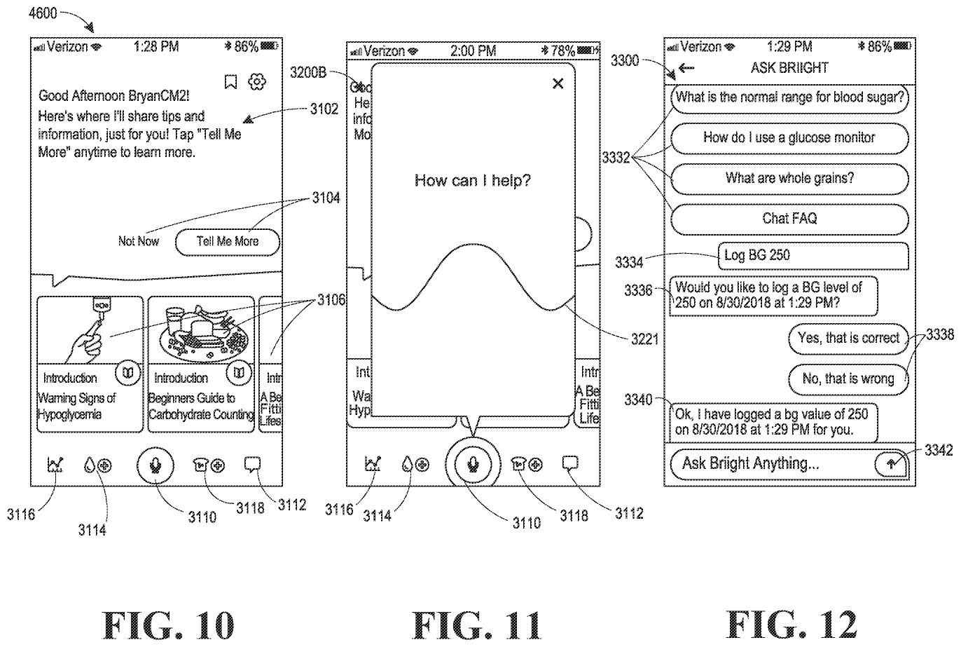

Example use of the system 100 will now be described with reference to the example screens shown in FIGS. 10, 11, and 12. FIG. 10 is an example screen 3100 of the interactive interface 122 of the IDM system 100 according to one embodiment. As illustrated, the screen 3100 represents a home screen or initial screen for the interactive interface 122. This screen 3100 can be the first to be displayed to the user upon accessing the system 100.

In this example, the screen 3100 includes an insight portion 3102. The insight portion 3102 can be configured to display insights to the user that are customized based on the user's previous interactions to the system 100. In some embodiments, the insights can include conversations or messages such as those described in the Example Reactive Events of Table 1. The insight portion 3102 can include user selectable options 3104 that allow a user to indicate whether he or she wishes to learn more about the offered insight. For example, the user selectable element 3104 can include a “Not Now” or a “Tell Me More” graphical indicia which may be selectable by the user. Pressing the “Tell Me More” graphical indicia would bring up additional data on the displayed subject, while selecting the “Not Now” graphical indicia may clear the screen. The additional data can include additional conversations, messages, or articles. In some embodiments, the “Tell Me More” graphical indicia can prompt the user to set personalized goals, for example, using the goal workflow described herein.

The screen 3100 also provides user-selectable options 3106 in the form of swipe cards that flow laterally from side to side on the displayed GUI and that allow a user to access content that has been selected for the user. Each card may display content that can include diabetes related information that has been customized for the user. Depressing each card on the touchscreen may activate the element 3106 and allow the users to move the cards from right to left, choosing which cards to become active on the display. In some embodiments, the cards show content which comprises customized learning workflows as described in the above.

As shown in FIG. 10, the screen 3100 also includes a voice input option 3110 located at the lower, center, portion of the GUI. A user may select the voice input option 3110 to input user voice data into the system 100. Upon selecting the voice input option 3110, screen 3200B of FIG. 11 may be displayed, and the system 100 may be configured to record user voice data, as will be described below. Entering user voice data may comprise, for example, recording an audio signal using a microphone on a user device. The audio signal may be processed by the natural language processor 132 so that spoken commands or questions contained therein are converted to a machine-understandable format for further processing by the system 100.

The screen 3100 in FIG. 10 also includes a text-based input option 3112. The user may select the text-based user input option 3112 to input text-based user data into the system 100. Text-based user data may comprise written data provided by the user. For example, a user can input written data using a keyboard on a user device. Upon selecting the text-based user input option 3112, screen 3300 of FIG. 12 may be displayed, and the system 100 may be configured to receive text-based user input, as will be described below. Text-based user input can processed by the natural language processor 132 so that commands or questions contained therein can be converted to a machine-understandable format for further processing by the system 100.

The screen 3100 also includes a blood glucose user input option 3114. The user may select the blood glucose user input option 3114 to input a blood glucose reading into the system. The screen 3100 also includes a data viewer user option 3116. The user may select the data viewer option 3116 to view user data, such as blood glucose data. In some embodiments, the data viewer user option 3116 may be used to access a screen 3400, as shown in FIG. 12, which displays blood glucose data.

FIG. 11 is an example screen 3200B of the interactive interface 122 illustrating a voice input function of the user interface 3020. In some embodiments, the voice input function is access by selecting the voice input option 3110 on the screen 3100 of FIG. 10. In some embodiments, the voice input function is configured to receive user voice input. The user voice input can be passed to the natural language processor 132 and response generator 134 of the interactive engine 130 as mentioned above. The natural language processor 132 and response generator 134 can parse the user voice input and generate responses that can be customized for the user.

As shown in FIG. 11, the screen 3200B can be configured to provide a visual indication that audio information is being recorded. For example, wave line 3221 can move in response to the audio signal being measured by a microphone of the user device to provide a visual indication of the recording. Similarly, in some embodiments, the voice input option 3110 can pulsate as an indication that audio information is being recorded.

In some embodiments, the voice input function can allow users to log data into the system 100. Such data can be stored as uploaded health data 144, for example. As one example, the user can select the voice input option 3110 and speak a command to log a blood glucose measurement. For example, the user can say “Log blood glucose 3400.” The natural language processor 132 can parse this input and understand that the user is entering a blood glucose measurement. The system 100 can then process the request, storing the blood glucose reading as user health data 144. This data will then available to the system 100 to further customize future interactions.

The voice input function can also be used to input and log other types of data as well. For example, a user can input data related to insulin injections, foods eaten, exercise performed, mood, stress, etc. In another example, the user can input data related to injection site location for insulin pens, patches, and continuous glucose monitoring devices. Injection site location data can be tracked so that the user can effectively rotate injection site location.

In some embodiments, the system 100 associates the voice input data with additional information known by the system 100, such as, for example, the date and time. This can facilitate tracking of the data.

FIG. 12 is an example screen 3300 of the interactive interface 122 illustrating a text-based response to a user voice input according to one embodiment. In some embodiments, after the user provides a user voice input, the interactive interface 122 can enter the text-based response screen 3300 to continue the interaction.

In some embodiments, the screen 3300 can show, for example, data 3332 from previous interactions. The screen 3300 can also show information related to the currently provided user voice data. For example, as illustrated, the screen 3300 shows a transcription 3334 of the provided user voice data. Continuing the blood glucose logging example described above, the transcription 3334 indicates that the user spoke “Log BG 3400.”

The screen 3300 can also include a text-based response 3336 to the input user voice data. In the illustrated example, response 3336 states: “Would you like to log a BG level of 3400 on Aug. 20, 2018 at 1:29 PM?” Thus, response 3336 can provide a confirmation of the provided user voice data. In some embodiments, the response 3336 can include other information. For example, the response 3336 can request additional information from the user.

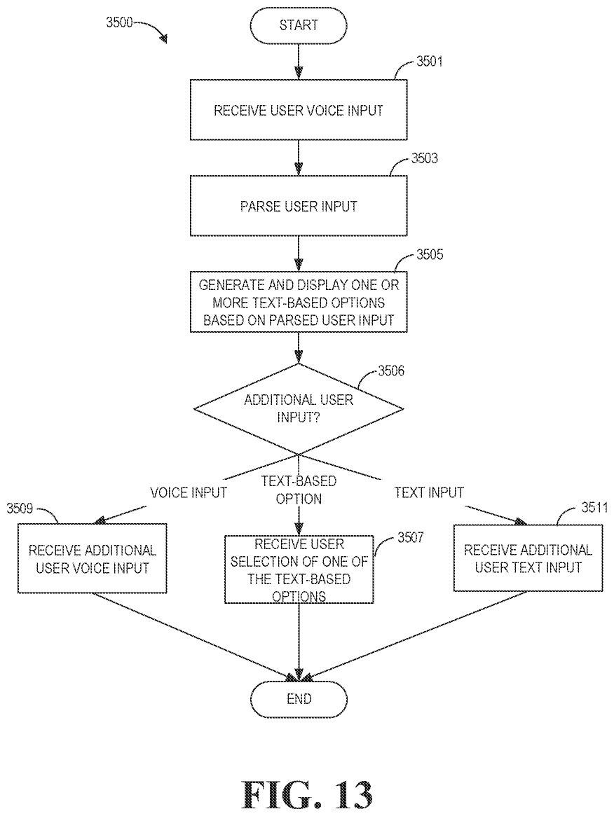

The screen 3300 can also include user-selectable options 3338. The user-selectable options 3338can be related to the response 3336. For example, as illustrated, user-selectable options 3338 of “Yes, that is correct” and “No, that is wrong” allow the user to quickly verify the response 3336. Providing user-selectable options 3338may streamline the interaction by providing the user with possible options that can be quickly and easily selected. The user-selectable options are described in more detail further below with reference to FIG. 13.

Finally, as shown in FIG. 12, upon selecting the user-selectable option 238 “Yes, that is correct,” the system 100 may provide a confirmation 3340 of the action taken. In the illustrated example, the confirmation 3340 indicates “Ok, I have logged a bg value of 3400 on Aug. 30, 2018 at 1:29 PM for you.”

FIG. 13 is a flow chart illustrating an embodiment of a method 3500 for a voice input module 3023 of an IDM system. The method 3500 begins at block 3501 at which user voice input is received by the system 100. In some embodiments, this occurs when the user selects the voice input option 3110 on the screen 3100 (FIG. 10) and speaks a command or question. The system 100 can record the user voice input and pass it to the interactive engine 130 for processing.

The method 3500 can then move to block 3503 at which the user voice input is parsed. In some embodiments, the natural language processor 132 (FIG. 1) parses the user voice input. This can include, for example, identifying spoken words and parsing the meaning thereof.

Next, at block 3505, the method 3500 generates and displays one or more text-based options to the user. The text-based options can be based on the parsed user voice input. The text-based options can be for example, the user-selectable options 238 displayed on the screen 3300 of FIG. 12.

In some embodiments, the text-based options provide the user with easily selectable options related to the question or command input by the user. For example, in the illustrated example of logging a blood glucose measurement, the options allow the user to quickly confirm or deny the measurement using user-selectable options provided on the screen.

In other embodiments, the text-based options can provide links to curated content related to the spoken command or question. For example, if the user asks about a particular food, the text-based options can include user-selectable links to recipes to related food, nutritional information, restaurants, etc.

Providing text-based options in response to the user's voice input data can streamline the process of interacting with the system 100 by predicting possible response and providing them to the user as easily selectable options.

From block 3505, the method 3500 moves to decision state 3506 at which is determined whether and which type of additional user input is received. From decision state 3506, the method 3500 can move to blocks 3507, 3509, or 3511 depending upon how the user responds. For example, at block 3507, the method 3500 can receive a user selection of one of the text-based options provided at block 3505. Alternatively, at block 3509, the method 3500 can receive an additional user voice input 3509, or at block 3511 the method 3500 can receive additional user text input.

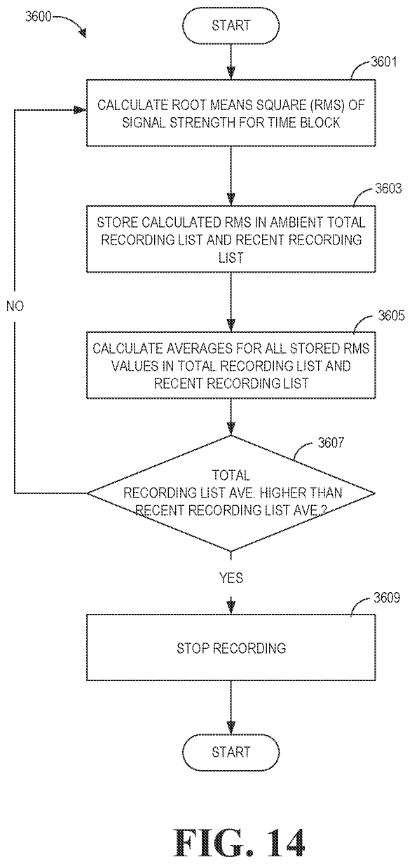

FIG. 14 is a flow chart illustrating an embodiment of another method 3600 for a voice input module 3023 of the IDM system 100. The method 3600 can be used, for example, by the natural language processor 132 to parse the voice input data at block 3603 of the method 3500 of FIG. 13. In some embodiments, the method 3600 can be used to determine when the user has finished providing voice input data. The method 3600 can be triggered when the user selects the voice input option 3110 (FIG. 10).

At block 3601, the method 3600 can include calculating the root means square (RMS) for the audio signal strength of an audio signal received during a time block. In one embodiment, the time block is 100, 200, 300, 400, 500, 600, 750, 1000, 2000 or 3000 ms, although other blocks both longer and shorter are possible.

At block 3603, the calculated RMS is stored in both an ambient total recording list and a recent recording list. In some embodiments, the ambient total recording list includes all calculated RMS values for each time block of the recording. In some embodiments, the recent recording list includes all calculated RMS values for each time block in a recent portion of the recording. In some embodiments, the recent portion of the recording includes the time blocks in the last 1.5 seconds of the recording, although other portions of the recording, both longer and shorter, can also be used.

At block 3605, an average RMS value for each of the total recording list and the recent recording list is calculated. At decision state 3607, the average RMS values for each of the total recording list and the recent recording list are compared against each other. If the average RMS value for the recent recording list is higher, the method 3600 continues by returning to block 3601. If the average RMS value for the total recording list is higher, the method 3600 moves to block 3609 at which the recording is ended.

As described above, an IDM system can include a user interface configured to interact, present or display information in a way to drive engagement with a user. The IDM system can be configured to deliver tailored engagement to the user in a manner configured to best help the user manage his or her disease. The tailored engagement can be based on, for example, stored user data, data received from various connected device (see FIG. 1), data entered by the user, stored content, etc. In some embodiments, the tailored engagement can be derived based at least in part on a user's previous interactions with the IDM system. To facilitate this engagement, the user interface of the IDM can include various modules. Certain modules are illustrated below with reference to example screen captures of an embodiment of an IDM. It should be appreciated that one or more of the modules can be included in and or executed by any of the IDM systems and/or user interfaces described above. Further, the following screen captures only provide examples and are not intended to be limiting of the disclosure.

### Example IDM System Methods

IDM systems, such as the IDM system 100 (FIG. 1) can implement various methods to facilitate disease management. In some embodiments, these methods are executed by the interactive engine 130. The methods may be involve the system 100 interacting or engaging with the user through the user interface 120. The methods can include accessing and storing various data in the user database 140 and content database 152.

An IDM system can include a goal module that can be configured to provide another mechanism of engagement between the user and the IDM system. Within the goal module, the user can be prompted with goals that the user can select and complete. In some embodiments, a list of categories of goals, a list of goals, and/or a level of difficulty of goals can be provided to the user to facilitate selection of a goal for completion. In some embodiments, one or more goals may be recommended to the user based on an initial assessment of the user. An initial assessment may be performed based on data previously collected from the user, such as fitness data, health data, or treatment adherence data. During the initial assessment, the IDM system may alternatively or additionally request information from the user for the determination of one or more initial goals, such as for example, areas of interest, strengths, and weaknesses of the user. Following the initial assessment, one or more categories of goals, goals, and/or levels of difficulty of goals can be recommended to the user.

The goals can be configured to match the user's current fitness and health level. As the user completes goals, more difficult goals can be suggested by the IDM system, which the user can select and complete. If a user fails to complete a goal, an easier goal can be selected and attempted.

In some embodiments, the goal module can include several categories of goals. Each category can include a number of goals of different difficulty levels. If a goal is completed, the goal module can recommend a new goal within the same category at a higher difficulty level or a new goal from a different category that may be of the same difficulty level or a higher or lower difficulty level. In some embodiments, if a goal is failed, the goal module can recommend a new goal within the same category at a lower difficulty level or a new goal from a different category that may be of the same difficulty level or a higher or lower difficulty level.

Table 2 depicts an example of goals of various difficulty levels within a “Blood Glucose” category of goals, ranging from level 1 (easiest) to level 4 (hardest). Table 2 shows an example duration for each goal and an example description that can be provided to the user.

TABLE 2

| Lvl. | Goal | Duration | Description |
|---|---|---|---|
| 1 | Read blood | 3 days | To reach this goal, just go to the next |
| | glucose | | screen and read the article. Then, for the |
| | article | | next 3 days, take a moment to think |
| | | | about what you learned. |
| 2 | Once-a-day | 3 times in | This goal will help you get in the habit |
| | blood sugar | a week | of checking your blood sugar. Check |
| | tracking | | your blood sugar 3 days this week at |
| | | | different times of the day. |
| 3 | Once-a-day | Daily for | Your goal is to check your blood sugar |
| | blood sugar | 7 days | before breakfast every day for a week. |
| | tracking | | Set yourself up for success by tracking |
| | | | your goal progress here! |
| 4 | Twice-a-day | Daily for | Nail this goal by checking your blood |
| | blood sugar | 7 days | sugar every morning and evening before |
| | tracking | | your meal. Set yourself up for success |
| | | | by tracking your goal progress here! |

Table 3 depicts an example of goals of various difficulty levels within an “Insulin” category of goals, ranging from level 1 (easiest) to level 4 (hardest). Table 3 shows an example duration for each goal and an example description that can be provided to the user.

TABLE 3

| Lvl. | Goal | Duration | Description |
|---|---|---|---|
| 1 | Read | 7 days | To reach this goal, just go to the next |
| | insulin | | screen and read one of the articles. Then, |
| | article | | for the next 7 days, take a moment to |
| | | | think about what you learned. |
| 2 | Take basal | Daily for | To meet this goal, remember to take your |
| | insulin on | 7 days | basal insulin as prescribed every day for |
| | time | | the next 7 days and log it in the app. |
| 3 | Rotate | 3 times in | To nail this goal, rotate your injection |
| | injection | a week | sites at least three times this week. |
| | sites | | |
| 4 | Take | Daily for | To meet this goal, remember your |
| | mealtime | 7 days | mealtime insulin as prescribed for the |
| | insulin on | | next 7 days, and log it in the app. |
| | time | | |

Table 4 depicts an example of goals of various difficulty levels within a first “Activity” category of goals, ranging from level 1 (easiest) to level 4 (hardest). Table 4 shows an example duration for each goal and an example description that can be provided to the user.

TABLE 4

| Lvl. | Goal | Duration | Description |
|---|---|---|---|
| 1 | Read | 7 days | To achieve this goal, just go to the next |
| | exercise | | screen to read one of the articles. Then, for |
| | article | | the next 7 days, take a moment each day |
| | | | to think about it. |
| 2 | Walk | 3 times in | To achieve this goal, walk 500 steps 3 |
| | 500 | a week | times this week. That'll take you 4-8 |
| | steps | | minutes, depending on your pace. |
| 3 | Walk | 4 times in | To nail this goal, walk 2,000 steps 4 times |
| | 2,000 | a week | this week. Depending on your pace, each |
| | steps | | session should be 25-30 minutes-but go |
| | | | at a speed that feels comfortable. |
| 4 | Walk | 4 times in | To master this goal, you'll need to walk |
| | 5,000 | a week | 5,000 steps 4 times this week. Depending |
| | steps | | on your pace, this will probably take you |
| | | | about an hour-but go at a speed that feels |
| | | | comfortable. |

Table 5 depicts an example of goals of various difficulty levels within a second “Activity” category of goals, ranging from level 1 (easiest) to level 4 (hardest). Table 5 shows an example duration for each goal and an example description that can be provided to the user.

TABLE 5

| Lvl. | Goal | Duration | Description |
|---|---|---|---|
| 1 | Read | 7 days | To achieve this goal, just go to the next |
| | exercise | | screen to read one of the articles. Then, |
| | article | | for the next 7 days, take a moment each |
| | | | day to think about what you learned. |
| 2 | Choosing the | 3 times in | To meet this goal, park far away from |
| | farthest | a week | your workplace, grocery store or place |
| | parking spot | | of worship 3 times this week so you'll |
| | | | have to walk a ways to the entrance. |
| 3 | Take the | 4 times in | To meet this goal, take the stairs instead |
| | stairs | a week | of an elevator or escalator 4 times this |
| | instead of | | week. |
| | an elevator | | |
| | or escalator | | |
| 4 | Talk a 10- | Daily for | To meet this goal, find time to fit a 10- |
| | minute walk | 7 days | minute walk into your day every day for |
| | | | 7 days. |

Table 6 depicts an example of goals of various difficulty levels within a “Nutrition” category of goals, ranging from level 1 (easiest) to level 4 (hardest). Table 6 shows an example duration for each goal and an example description that can be provided to the user.

TABLE 6

| Lvl. | Goal | Duration | Description |
|---|---|---|---|
| 1 | Read | 7 days | To reach this goal, just go to the next |
| | healthy | | screen and read one of the articles. Then, |
| | eating | | every day this week, take a moment to |
| | article | | think about what you've learned. |
| 2 | Make one | 7 days | To reach this goal, make one of the |
| | new | | delicious, diabetes-friendly recipes on the |
| | healthy | | next screen. And there are more to choose |
| | recipe | | from in the app: Just chat with me and I |
| | | | can help you find them. |
| 3 | Make | 3 times in | To reach this goal, swipe through our |
| | healthy | a week | guide to healthy food swaps on the next |
| | food | | screen and make a healthy food swap 3 |
| | sways | | times this week. |
| 4 | Keep a | 7 days | To reach this goal, jot down everything |
| | food diary | | you eat and drink for the next week in a |
| | | | notebook, or put it in a notes program on |
| | | | your smartphone. (Don't forget sneaky |
| | | | items like salad dressing and coffee |
| | | | creamer.) |

Table 7 depicts an example of goals of various difficulty levels within an “Risk Reduction” category of goals, ranging from level 1 (easiest) to level 4 (hardest). Table 7 shows an example duration for each goal and an example description that can be provided to the user.

TABLE 7

| Lvl. | Goal | Duration | Description |
|---|---|---|---|
| 1 | Read diabetes | 7 days | To achieve this goal, just go to the |
| | health risk | | next screen and read one of the |
| | article | | articles. Then, for the next 7 days, |
| | | | take a moment each day to think |
| | | | about what you learned. |
| 2 | Check feet | 3 times in | To achieve this goal, check your feet |
| | for blisters, | a week | for blisters, sores or swollen areas |
| | sores, or | | 3 times this week. |
| | swollen areas | | |
| 3 | Practice | Daily for | To achieve this goal, just go to the |
| | stress | 7 days | next screen and read one of the |
| | reduction | | articles. Then practice what you |
| | techniques | | learned every day this week. |
| 4 | Schedule an | 7 days | To achieve this goal, tap the link on |
| | appointment | | the next screen to make an |
| | with a | | appointment with a certified diabetes |
| | diabetes | | educator. |
| | educator | | |

The IDM system can monitor progress and engage with the user during performance of a goal to enhance adherence to the goal or determine issues related to the goal experienced by the user. For example, the IDM can provide addition content to user, such as articles or recipes, related to the goal. In some embodiments, the IDM can provide recommendations to the user for completion of the goal or ask questions to the user regarding progress towards the goal. In some embodiments, the IDM module can provide additional content and/or recommendations based on the user's answers and/or progress.

The IDM system can also generate content in other modules based on the goals that user is pursuing in the goal module. For example, if the user is pursuing a goal related to physical activity, a learning plan related to physical activity can be suggested in the learn module. Similarly, if a user is pursuing a goal related to diet, a learning plan relating to diet can be presented in the learn module.

If a user fails to complete a goal, the IDM system can engage with the user to try and figure out why the user did not complete the goal. For example, the user can be prompted with an assessment to determine the user's feelings about the goal. The results of the assessment can be used to derive new goals that are configured to drive engagement between the user and the system. The goal module can modify goals based on the user's past experiences in the goal module as well as in other parts of the user interface of the IDM system. In some embodiments, if a user fails to complete a goal, the initial assessment can be repeated. The results of repeated initial assessment, either alone, or in combination with the users past experiences in the goal module, past experiences in other parts of the user interface of the IDM system, and/or the results of the assessment of the user's feelings about the previous goal, can be used to determine a recommendation of one or more goals to the user.

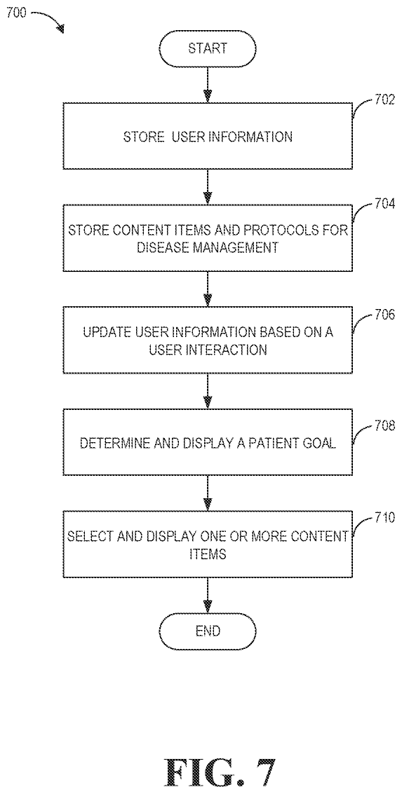

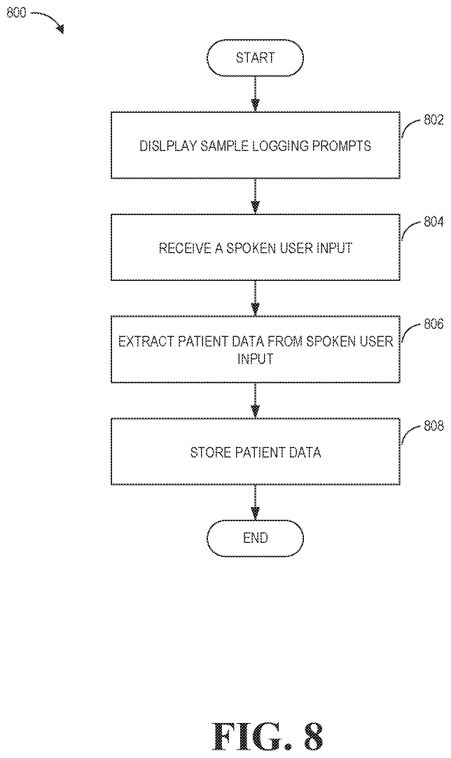

FIG. 7 is a flowchart illustrating an example process 700 for determining a patient goal or goals in an integrated disease management system. The process 700 begins at a start step. Next, the process moves to a step 702, at which the system stores user information related to a patient having a disease. User information can be stored in a user database. The user information can include at least one of measured patient disease management data and user-inputted patient disease management data. Measured patient disease management data can be data received from an external device, such as any of the devices shown in FIG. 1 as connected to the IDM 100. For example, the measured patient disease management data can be received from a smart diabetes monitor, a smart insulin pen, a smart insulin pump, and a fitness tracker. User-inputted patient disease management data can be similar data that the user has entered through the IDM system. Such user-inputted patient disease management data can be entered, for example, using the logging method described below with reference to FIG. 8. The user data can be data related to the patient's disease. In an example, where the disease is diabetes. The user data can be data related to blood glucose, insulin injections, diet, physical activity, etc. In some embodiments, the user-inputted patient disease management data can include data collected during an initial assessment by the goals module.

At a step 704, the IDM system stores content items related to recommended lifestyle choices for improving patient outcomes and protocols for disease management. Content items may be stored in a content database. Content related to recommend lifestyle choices for improving patient outcomes can include, for example, content curated to help the user manage his or her disease. This can include for example, curated courses or information on managing injections, information related to diet, information related to exercise, etc. Protocols for disease management can include protocols that determine how to improve the user's disease status. For example, if the user is experiencing high blood sugar, a protocol can be provided with parameters that define steps that can be taken to lower the user's blood sugar. Protocols can be developed by medical professionals, such as CDEs.

Next, at a step 706, the system updates the user information in the user database based on a user interaction with an interactive user interface for providing integrated disease management. For example, when the user engages with the IDM system, this interaction may cause the system to store additional information about the user in the user database. Such information can be used to tailor future interactions with the system. In some embodiments, the user interaction can be at least one of the user providing user-inputted patient disease management data with the interactive interface and the user providing measured patient disease management data from one or more patient monitoring devices. This can include the user manually entering data, or the IDM system receiving data automatically from a smart, connected device. In some embodiments, this can include data provided during an initial assessment by the goals module.

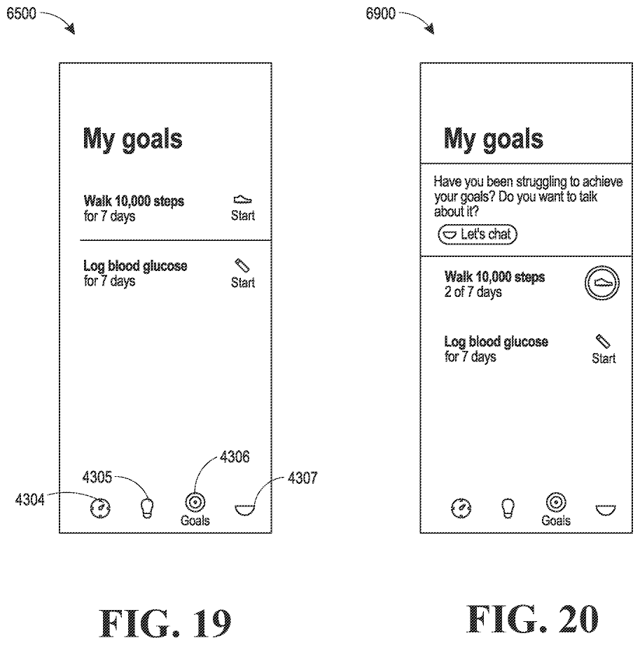

At a step 708, the system determines a patient goal related to improving disease management based on the user information and the stored protocols for disease management and displaying the patient goal to the user on the interactive user interface. The system may analyze the user information to determine a goal that will be helpful to the user for managing his or her disease. The determination can be based on the stored protocols as well as previously entered user data, such as data entered during an initial assessment by the goals module. The system can determine a goal that is “within the patient's reach” based on knowledge of the user from past interactions between the system and the user. Example goal module, displaying goals to a user and interacting with the user are shown in FIGS. 19-22 and 33-39, described below.

In some embodiments, the system can determine a plurality of patient goals to display to the user to allow the user to select a patient goal. In some embodiments, the system can determine a category of patient goals, such as for example blood glucose goals, insulin goals, exercise or activity goals, nutrition goals, or risk reduction goals, to display to the user to allow the user to select a category of goals prior to determining the patient goal. In some embodiments, the system can determine a difficulty level of a goal to display to the user.

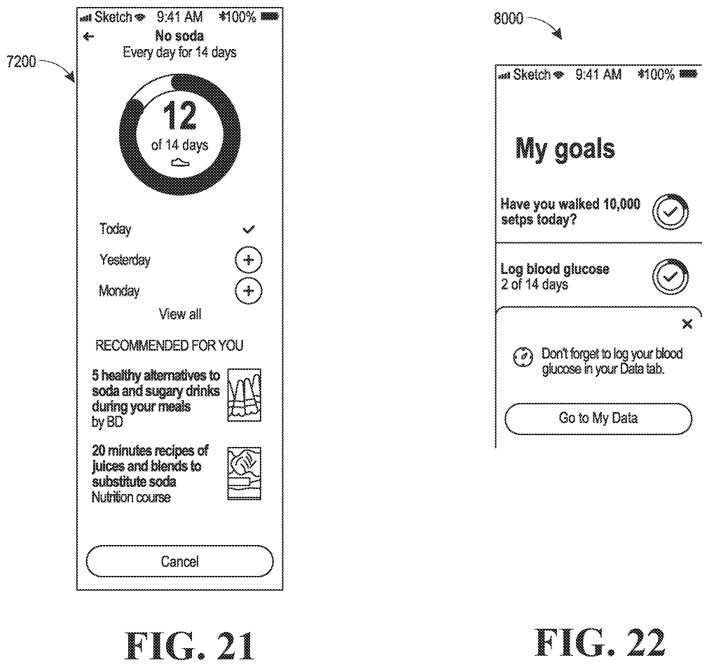

At a step 710, the system can also select one or more content items from the content database based on at least the determined patient goal and the user information and display the selected one or more content items to the user on the interactive user interface. Thus, in addition to providing a recommended goal the user, the system may provide related content to the user as well. An example is shown in FIG. 21.

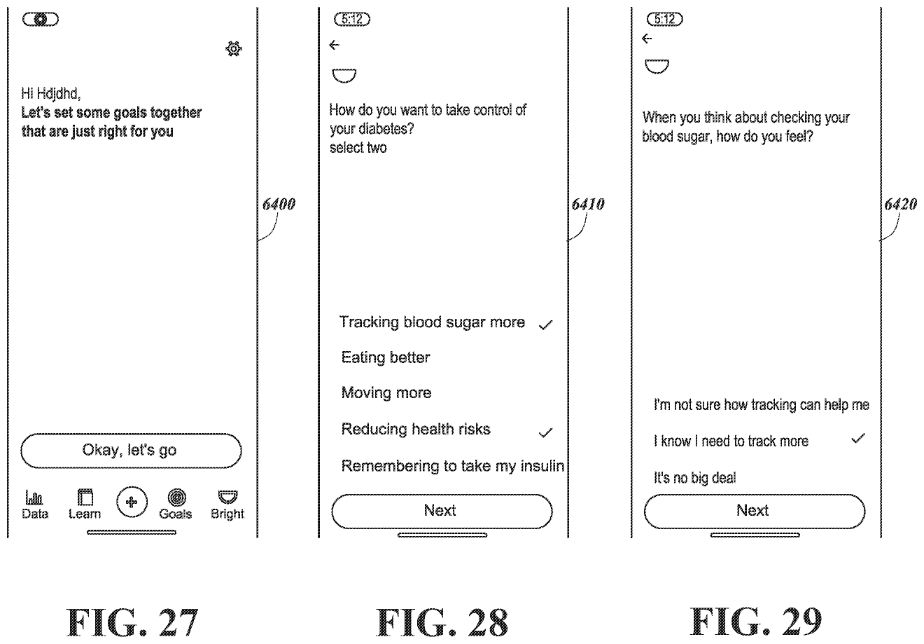

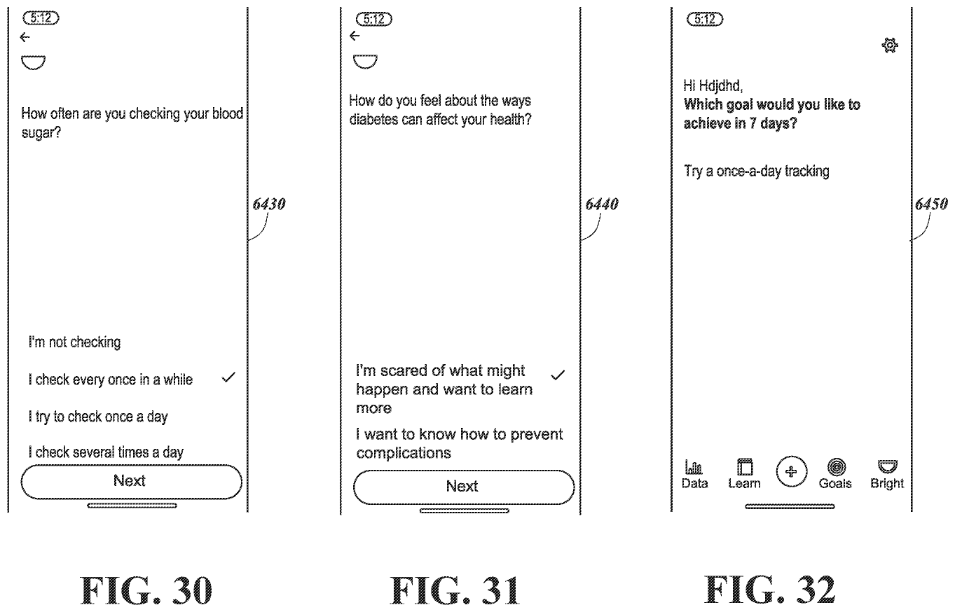

FIGS. 27-32 are example screen captures of a user interface to a goal module of an IDM system depicting an example of an initial assessment workflow for determining and providing a goal recommendation to a user.

FIG. 27 shows an example screen 6400 of the user interface to a goal module according to one embodiment. The screen 6400 includes a prompt to the user to begin the initial assessment with the text “Let's set some goals together that are just right for you.” The screen 6400 further includes a selectable option to begin the initial assessment labeled “Okay, let's go.”

After the user decides to begin an assessment the goal module can ask a series of questions to the user and allow the user to select responses as shown on example screens 6410, 6420, 6430, 6440 of FIGS. 28, 29, 30, and 31, respectively. Screen 6410 of FIG. 28 shows that the user has selected options of “Tracking blood sugar more” and “Reducing health risks” in response to the question “How do you want to take control of your diabetes?” Screen 6420 shows that the user has selected the option of “I know I need to track more” in response to the question “When you think about checking your blood sugar, how do you feel?” Screen 6430 shows that the user has selected the option of “I check every once in a while” in response to the question “How often are you checking your blood sugar?” Screen 6440 shows that the user has selected the option of “I'm scared of what might happen and want to learn more” in response to the question “How do you feel about the ways diabetes can affect your health?”

After the user answers questions from the goal module, a list of recommended goals that can be selected by the user are displayed on a screen 6450, as shown in FIG. 32. The screen 6450 of the example workflow lists a selectable goal of “Try once-a-day tracking” which can be based at least in part on the user's answers to the questions of screens 6410, 6420, 6430, and 6440.

FIG. 8 is a flowchart illustrating an example process 800 for logging patient data in an integrated disease management system. The process 800 can be implemented by a logging module that can provide the user with a quick and efficient method for logging diabetes care related information. As will be shown, in some embodiments, the logging module may utilize voice logging. The voice logging may provide a number of sample log prompts, including blanks that the user can easily fill in.

The process 800 begins at a start step and moves to a step 802 at which the system displays a plurality of sample logging prompts. The sample logging prompts can be displayed on an interactive user interface. Each of the sample logging prompts can include a phrase relating to a type of patient data associated with a disease of the user and including at least one blank. The sample logging prompts can help guide the user in understanding how to log data with the IDM system, and help the user understand the types of data that can be logged. FIG. 24, described below, illustrates several sample logging prompts in the context of diabetes.

The sample logging prompts can be based at least in part on the disease of the user and previously stored patient data. For example, the system can understand which type of data is useful for treating the disease as well as which types of data the user has entered in the past to determine the sample logging prompts. In the case of diabetes, for example, the sample logging prompts can be related to are related to one or more of blood glucose measurement, insulin dosing, diet, and physical activity

At a step 804, the system receives a spoken user input. The spoken user input can be recorded with a microphone of a user device. The spoken user input can include the user verbally repeating one the sample logging prompts with patient data inserted into the at least one blank. Receiving the spoken user input can include parsing an audio signal using the method of FIG. 14, described above.

At a step 806, the system can extract the patient data from the spoken user input with a natural language processor. This can include interpreting the spoken user input and translating the spoken user input into a computer-readable format.

At a step 808, stores the patient data in a user database of the integrated disease management system. In this way, the user can simply and quickly use vocal commands to log patient data into the system

In some embodiments of the process 800, the system, after receipt of the spoken user input, removes the displayed sample logging prompt associated with the spoken user input from the display and displays a new sample logging prompt to replace the removed sample logging prompt. This can encourage the user to continue logging data as additional prompts are provided. In some embodiments, the system also displays the text of the spoken user input to the user. This can allow the user to verify that the system has understood correctly. The system may also prompt the user to confirm that the data is correct.

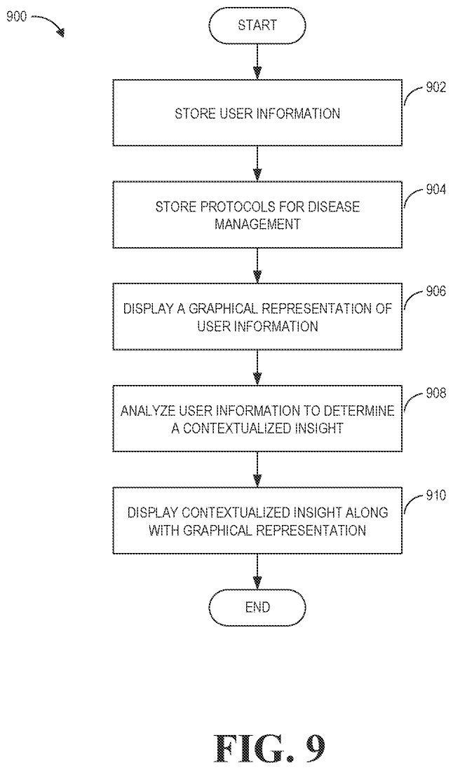

FIG. 9 is a flowchart illustrating an example process 900 for displaying contextualized insights along with a graphical representation of patient data in an integrated disease management system. The system can analyze the data displayed to the user and provide beneficial, contextualized insights that can help the user to understand and apply the data.

The process 900 begins at a start step and then moves to a step 902, at which the system stores user information related to a patient having a disease. The user information can be stored in the user database. The user information can include at least one of measured patient disease management data and user-inputted patient disease management data. Measured patient disease management data can include data received from one or more patient monitoring devices. The one or more patient monitoring devices can be, for example, a smart diabetes monitor, a smart insulin pen, a smart insulin pump, and a fitness tracker or others. User-inputted patient disease management data can be data entered by the user.

At a step 904, the system stores, in a content database, protocols for disease management. The protocols can provide steps for managing a user's disease as described above. At a step 906, the system a graphical representation of at least a portion of the stored user information. The graphical representation can be for example, one or more graphs or plots of patient data for a given time period such as a day, a week, or a month.

At a step 908, the system analyzes the at least a portion of stored user information displayed on the interactive display based at least in part on the protocols for disease management to determine a contextualized insight related to the at least a portion of stored user information. The system can determine trends in the displayed data that may not be readily apparent to the user and provide insights regarding these trends so as to help the user manage the disease.

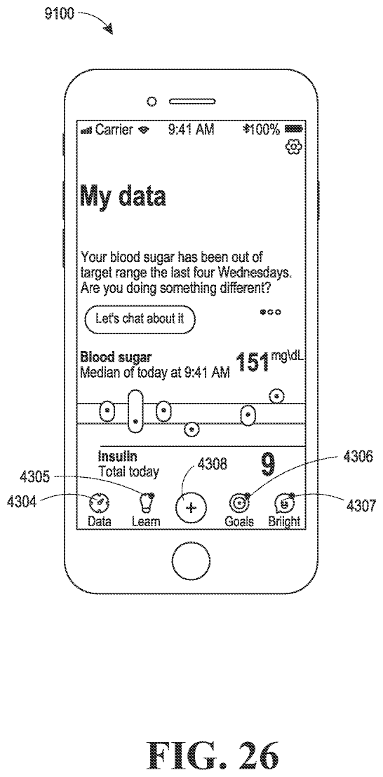

At a step 910, the system displays, on the interactive display, the contextualized insight along with the graphical representation. An example of this feature is shown in FIG. 26, described below. The process 900 can be helpful because it can allow a user to understand and apply their patient data in a way that may not readily apparent to the user based on the patient data alone.

### Example IDM System Screens

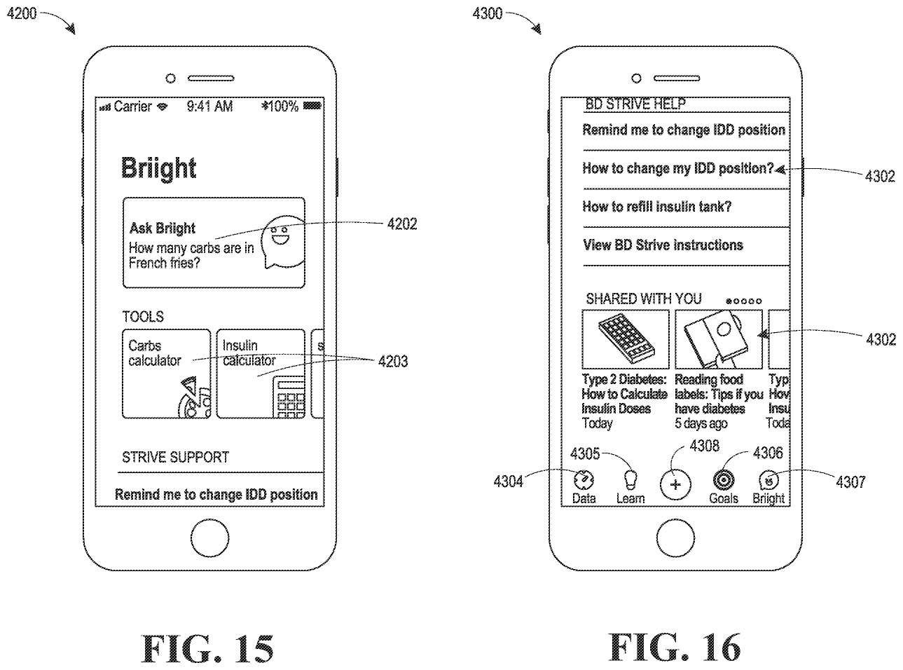

FIGS. 15 and 16 are example screen captures of home screen of a user interface of an IDM system according to an embodiment. The home screen can be presented to the user after the user has completed an onboarding module or when the user first accesses the IDM system after having completed the onboarding module. The home screen can present the user with information and provide links for accessing various other modules of the IDM system.

FIG. 15 shows an initial example of a home screen 4200. As illustrated, the screen 4200 includes a user-selectable button 4202 labeled “Ask Briight.” The user-selectable button 4202 can also be labeled differently in other examples. The user-selectable button 4202 can be accessed to allow the user to access an interactive portion of the user interface. For example, the user-selectable button 4202 can be used to access a chatbot, which as described above can allow the user to interact with the user interface in a natural language fashion. For example, the user can interact with the user interface by typing natural language questions or by speaking natural language questions verbally to the system after selecting the user-selectable button 4202. In the illustrated example, the user-selectable button includes a sample of the type of question that can be asked to the system. As illustrated, the sample is “How many carbs are in French fries?” By providing the user with the sample, the IDM system may intuitively prompt the user to understand which types of questions can be asked to the system after selecting the user-depressible button 4202. Other samples can be included or the sample can be omitted.

In the illustrated example, the screen 4200 also includes a series of selectable cards that can be selected to access various tools available to the user in the IDM system. For example, as illustrated, cards for “Carbs calculator” and “Insulin calculator” are presented. In some instances, cards for frequently access tools may be displayed. In some environments, access to tools may be provided in other ways such as drop down menus, user-selectable buttons, etc.

FIG. 16 presents an additional example of a home screen 4300. In some embodiments, the home screen 4300 can be accessed by scrolling down from the screen 4200 of FIG. 15. As shown, the screen 4300 may include links 4302 for accessing certain content within the IDM system. For example, the links 4302 may be used access frequently used articles or tutorials. In the illustrated example, links for “Remind me to change IDD position,” “How to change my IDD position?,” “How to refill insulin tank?,” and view “BD Strive Instructions.” Accessing any of the links 4302 can take the user immediately to the selected content.

As shown, the screen 4300 also includes additional content 4303 for the user. Links to content 4303 “Type 2 Diabetes: How to Calculate Insulin Doses” and “Reading Food Labels: Tips If You Have Diabetes” are presented. The content 4303 can be tailored for the user. For example, the IDM system may select specific content based on the user's past experiences with the IDM system and display links to the content directly on the home screen 4300. The content 4303 may change over time, for example, as the system learns more about the user's preferences and as the user has more experiences with the system.

As shown on the screen 4300, the home screen may include a menu with different icons for accessing different modules of the IDM system. As illustrated, the screen 4300 includes an icon 4304 for accessing a data module, an icon 4305 for accessing a learn module, an icon 4306 for accessing a goals module, an icon 4307 for accessing a chatbot module, and an icon 4308 for entering user data with a logging module. Example screens for each of these modules are shown and described below.

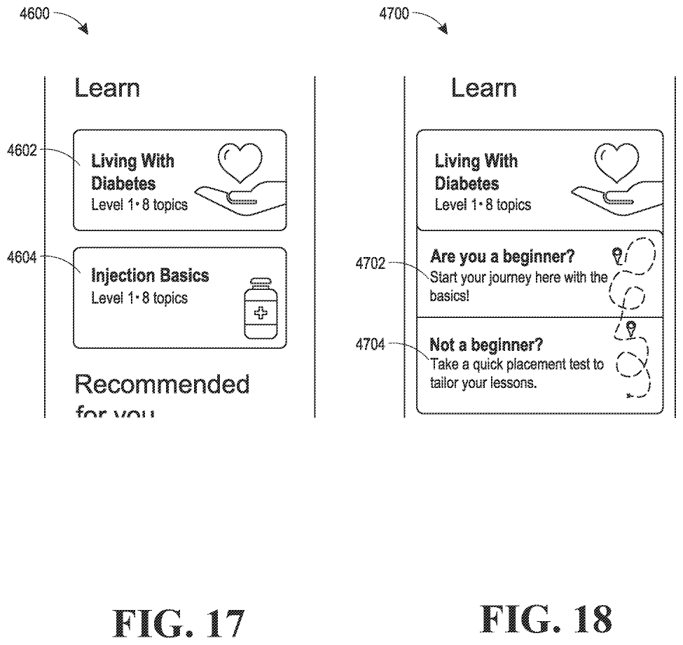

FIGS. 17 and 18 are example screen captures of a learn module of a user interface of an IDM system according to an embodiment. The learn module may be accessed, in some examples, by selecting the icon 4305 on the home screen (see FIG. 16). The learn module can be configured to provide customized or tailored curriculum or learning plans for the user. The curriculum can be selected and curated based on the user's past interactions with the system. The curriculum can be selected based on the user's level of knowledge and comfort with various topics. The learn module can provide the user with context specific insights and profile specific curriculum. The content provide by the learn module may be determined at least in part, by the information in the user's profile and the rules described above (see, for example, FIGS. 21-29 and related text). Further, at the end of a piece of curriculum/interaction the learn module can engage the user with behavioral conversation (e.g., to assess the user's comfort level with the material, which is a psychological indicator of success) to guide future content.

FIG. 16 presents an initial screen 4600 of the learn module. As shown the screen 4600 can present the user with one or more learning plans. In the illustrated example, a first learning plan 4602, entitled “Living with Diabetes,” and a second learning plan 4604, entitled “Injection Basics,” are presented to the user. The user may access either of the learning plans 4602, 4604 by selecting them on the screen 4600. The learning plans 4602, 4604 shown on the screen 4600 are only examples of learning plans. Various other learning plans can be provided to the user on the screen 4600. As will be described in more detail below, a learning plan can comprise a guided curriculum that can be customized for the user. For example, a learning plan can be configured to teach material to a user in a manner that is best suited to the users learning style and knowledge base.

The screen 4600 may display learning plans that are recommended for the user by the system. For example, the learning plans 4602, 4604 shown in FIG. 16 relate to the basics of diabetes care. These learning plans may be presented to a new user or to a user that is unfamiliar with the basics of diabetes care. A user with more experience with the IDM system or with more knowledge of diabetes care may be presented with more complex learning plans that are more suited to that user's knowledge base. As noted previously, the system may customize content based on the user's profile and the user's past experiences with the system.

FIG. 18 illustrates an example screen 4700 of the learn module. The screen 4700 may be displayed after the user selects the learning plan 4602, “Living with Diabetes,” from the screen 4600 of FIG. 16. As shown, in the illustrated example, the screen 4700 presents the user with two options related to the selected learning plan. Specifically, in the illustrated example, the user is presented with a beginner option 4702 and a not-a-beginner option 4704. The options 4702, 4704 allow the user to indicate their familiarity with the material. For example, if the user is new to living with diabetes, the user may select the beginner option 4702. As Illustrated, the beginner option asks, “Are you a beginner?” Start your journey here with the basics!” If the user selects the option 4702, the user can be guided to more beginner material. For example, if the user selects the option 4702, the user may begin at the very beginning of the learning plan. The not-a-beginner option 4704 asks, “Not a beginner?” Take a quick placement test to tailor your lessons.” This option 4704 may be selected by users who already have some familiarity with the material of the learning plan. Selection of the option 4704 may take the user to a placement test to determine the user's familiarity with the material. Based on the outcome of the placement test, the user may be inserted into the learning plan at various points that correspond to the user's familiarity with the material.

In many cases, the user will move through the lessons sequentially before moving on to the next course. However, based on the interactions with the learning plan, the learn module may customize the learning plan by moving the user through the course in a different order to best suit the user's learning style and knowledge base. FIG. 6, described above, is a flow chart illustrating example movement of a user through a learning plan. The learn module can pose questions that may be configured to assess the user's comfort and knowledge related to the learning plan so as to place the user into the learning plan at the spot that best matches the user's current knowledge and experience. As a result of the assessment, the user may be placed into the middle of a learning plan. If the initial assessment reveals that the user is already familiar with the material, then this information can be inserted into the learning plan at this point or at any suitable point based on the assessment. In this example, the user has passed the introduction and preparation courses without needing to take the additional course material based on the initial assessment.

FIGS. 19, 20, 21, and 22 are example screen captures of a user interface to a goal module of an IDM system. FIG. 19 shows an example screen 6500 of a goals module according to an embodiment. The screen 6500 can be configured to display possible goals to a user. The possible goals can be suggested by the IDM system. The goals can be suggested by the IDM system based at least in part on, for example, the user's profile and the user's past interactions with the IDM system. In some embodiments, the goals can be suggested based on an initial assessment conducted by the goal module, as described herein. As illustrated, two possible goals are displayed on the screen 6500. A first example goal states “Walk 10,000 steps for 7 days.” The system may suggest this goal based on the user's known activity level based on interactions with the system (e.g., previous user data inputs) or data received from connected devices, such as step counters of fitness trackers. For example, the goal module may suggest a step goal that is, for example, 10%, 20%, 25%, or 30% higher than a number of steps that the user has averaged over the past day, week, or month. Other metrics for determining the step goal are also possible (e.g., calories burned, exercise minutes, etc.). Where the user profile does not include past activity data on which to base the goal, the goal module may suggest a moderate goal based on, for example, a scientific recommended daily step-count.

As shown in FIG. 19, the screen 6500 includes a second suggested goal of “log blood glucose for 7 days.” Although two suggested goals are shown on the screen 6500, other numbers of suggested goals may be included in other embodiments. Further, the illustrated goals are provided for example only. Other goals may also be included.

For each suggested goal, the screen 6500 can include a start button that the user can select if they wish to try the goal. Additionally, the screen 6500 can include a menu with icons that allow the user to select additional modules of the user interface. For example, the menu includes the icon 4304 for accessing the data module, the icon 4305 for accessing the learn module, the icon 4306 for accessing the goals module, and the icon 4307 for accessing the chatbot module. These icons may also appear on the home screen 4300, as shown in FIG. 16, described above. These icons can allow for quick and easy access to other modules directly from within the goal module.

FIG. 20 illustrates an example screen 6900 that can be displayed if the user is not meeting his or her goals. As shown, the system may prompt the user to inquire why the user has not met the goal. For example, the screen 6900 asks, “Have you been struggling to achieve your goals? Do you want to talk about it? Let's chat.” Selecting the let's chat option may bring the user to the chatbot interface. The IDM system may then inquire about why the user has not been able to meet the goal. The user may respond either written or orally to the system. In this way, the goals module can receive feedback about why the user has not met the goals. Such feedback may be used to adjust the goals going forward. This system may create a more customized and tailored experience for the user that may help the user to achieve his or her goals.

While FIG. 20 illustrates one example of a prompt from the system when a user is not meeting his or her goals, other prompts may be initiated during an attempt to complete a goal. For example, prompts to chat with the IDM system may be a) initiated at predetermined times within the duration of the goal, b) in response to meeting a goal milestone, such as for example, walking 10,000 steps in one day for a goal of “Walk 10,000 steps for 7 days,” or c) based on other measured or user-inputted data received by the IDM. Additionally, an option to chat may be presented to the user throughout the duration of a goal to facilitate questions or feedback from the user.

FIG. 21 illustrates an example screen 7200 for tracking a “no soda every day for 14 days goal.” As shown, the user is on day 12 of 14. A status indicator circle indicates how close the user is to complete in the goal. In this example, below the status indicator the user has the option to enter whether they completed the goal for each day. As illustrated by a checkmark, the user has completed the goal for today. In this example, the user has not indicated that they have completed the goal for yesterday or Monday. However, they may still enter that they completed the goal on this day by selecting the plus icon associated with the day.

Below the goal tracking portion of the screen 6700, the goal module may include a portion of the screen 6700 for displaying content to the user. The content can be related to the goal being tracked. In this example, the goal relates to not drinking soda and the display content includes an article for “5 healthy alternatives to soda and sugary drinks during your meals” and an article for “20 minute recipes of juices and blends to substitute soda.” Because the user is currently pursuing a goal related to not drinking soda, the content related to alternatives to soda may be highly relevant to the user. Thus, it may be likely that the user may select the article to read the content. In some embodiments, the additional content displayed to the user can be displayed a) at a predetermined time within the duration of the goal, b) based on the user's progress towards completing the goal, c) in response to a request from the user, or d) in response to comments from the user during a chat with the IDM system.

FIG. 22 illustrates an example screen 8000 that displays the user's goals. The screen 8000 also includes an example of a notification that has popped up to remind a user to record user inputs into the system. In the illustrated example, the notification states “Don't forget to log your blood glucose in your data tab.” Thus, when the user is in the goal module, the IDM system may prompt the user to access additional modules, such as the data logging module, by providing the user with a notification, for example as shown in FIG. 22. Such notifications can be provided to the user while the user is accessing any of the modules.

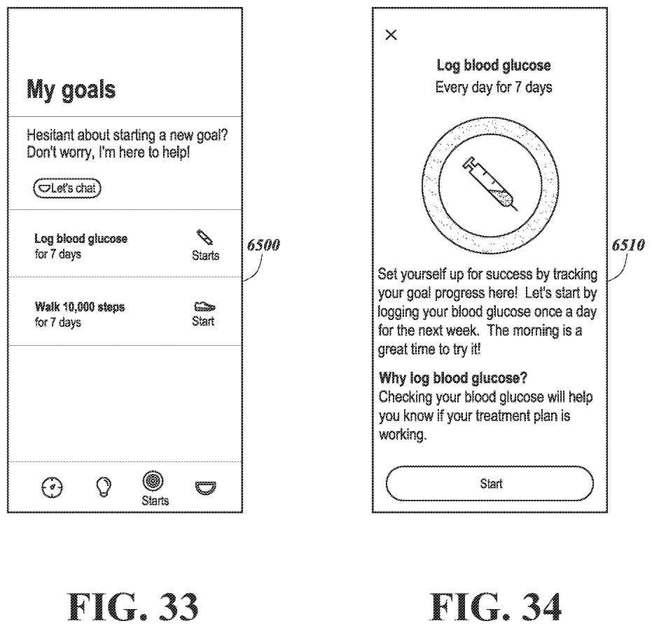

FIGS. 33-39 are example screen captures of a user interface to a goal module of an IDM system depicting an example workflow for a goal of “Log blood glucose for 7 days.”

FIG. 33 shows an example screen 6500 of a goals module according to an embodiment. As illustrated, two possible goals are displayed on the screen 6500. A first example goal states “Walk 10,000 steps for 7 days, and a second goal states “Log blood glucose for 7 days.” As described herein, for each goal, the screen 6500 can include a start button that the user can select if they wish to try the goal. For the example workflow depicted in FIGS. 33-39, the goal “Log blood glucose for 7 days” is selected.

After selection of the goal “Log blood glucose for 7 days,” the goal module can display a screen 6510, as shown in FIG. 34. The screen 6510 includes status indicator circle indicating how close the user is to completing the goal. As shown in FIG. 34, the status indicator circle shows no current progress towards the goal. The screen 6510 also includes a description of the goal and an explanation of the relevance of the goal. The screen 6510 further includes a start button that the user can select to begin the goal.

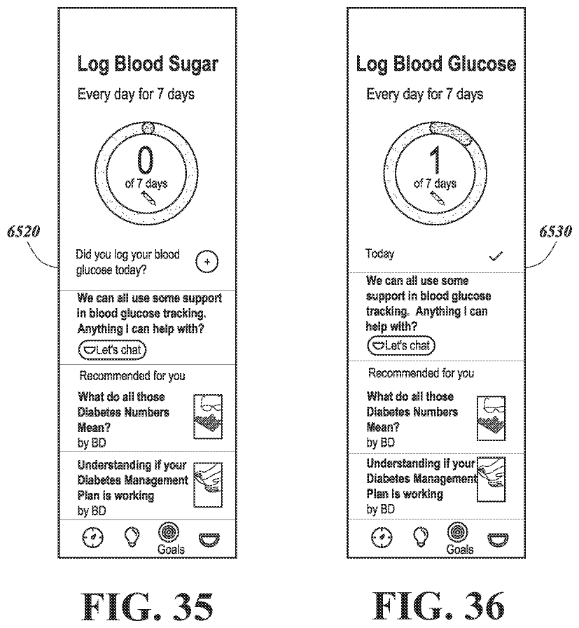

After selection of the start button of the screen 6510, the goal module can display a screen 6520, as shown in FIG. 35. The status indicator circle on the screen 6520 indicates that the goal has been initiated but that no progress has been made. The user is on day 0 of 7. Below the status indicator is an option for the user to enter whether they completed the goal for each day. The plus sign indicates that the user has not yet indicated that they have completed the goal of logging blood glucose for the day. Below, the option for the user to enter whether they completed the goal for each day, the screen 6520 includes a chat prompt. The chat prompt asks “We can all use some support in blood glucose tracking. Anything I can help you with?” Selecting the “let's chat” option below the chat prompt can bring the user to the chatbot interface, as described herein. In some embodiments, the chat prompt may be present throughout the duration of the goal. In other embodiments, the chat prompt may appear only at certain times or may change based on the user's progress within the goal. Below the chat prompt, the screen 6520 includes a portion of the screen for displaying content to the user. The content can be related to the goal being tracked. In this example, the display content includes an article for “What do all those Diabetes Numbers Mean?” and an article for “Understanding if Your Diabetes Management Plan is Working.”

After the user enters that the goal has been complete for the first day, the goal module can display a screen 6530, as shown in FIG. 36. The status indicator circle on the screen 6530 indicates that the user has completed day 1 of 7. Below the status indicator, the checkmark indicates that the user has completed the goal for today.

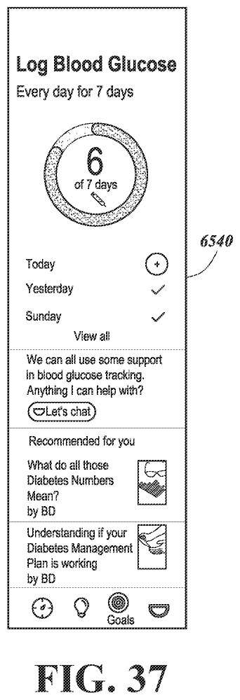

FIG. 37 shows an example screen 6540 of a goal module after the user has completed 6 of 7 days of the goal. As demonstrated by the checkmarks next to the options for indicating goal completion, the user completed the goal yesterday and Sunday. The plus sign indicates that the user has not yet completed the goal today.

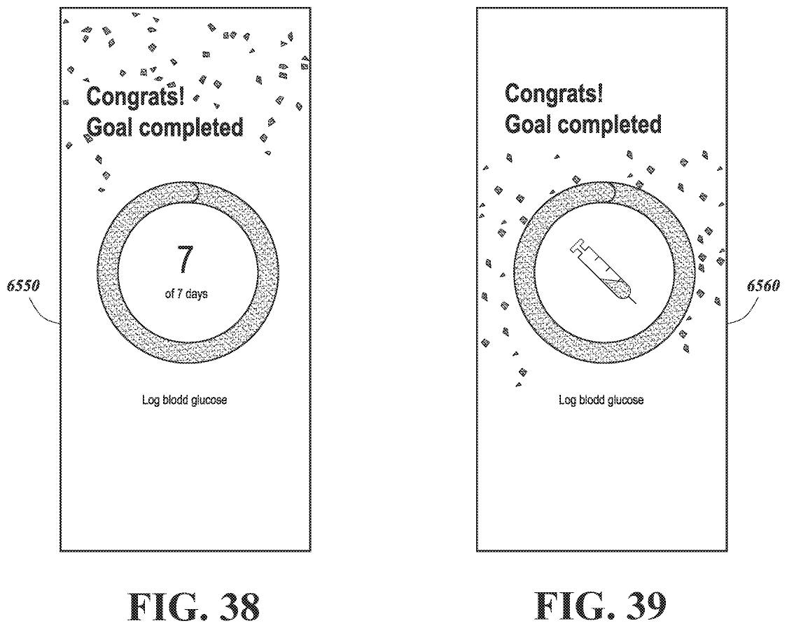

After the user indicates that the goal has been completed for today on screen 6540, the goal module can display screen 6550, as shown in FIG. 38. The screen 6550 shows a congratulatory message stating “Congrats! Goal completed.” The status indicator circle on the screen 6550 indicates that the user has completed day 7 of 7. The screen 6550 further shows an animation to indicate successful completion of the goal. In the example of screen 6550, the animation is an animation of confetti.

After the screen 6550 has been displayed, the goal module can display the screen 6560, as shown in FIG. 39. The screen 6560 also shows an animation of confetti to indicate a congratulations or celebration to the user for completing a goal. In comparison to the screen 6550, the screen 6550 has replaced the text stating “7 of 7 days” with an icon representing the goal. On the screen 6550, the icon is a syringe.

After a goal has been completed or failed, the goal module can recommend a new goal as described herein.

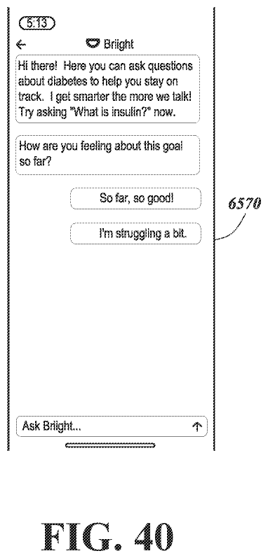

FIG. 40 is an example screen capture 6570 of a chatbot interface based on a user selection during use of the goal module, such as, for example, during the workflow shown in FIGS. 33-39. If a user selects to initiate a chat, for example by selecting the “let's chat option” of the screen 6570, the chatbot interface may display a question related to the goal such as “How are you feeling about the goal so far?” with optional user responses such as “So far, so good!” and “I′m struggling a bit,” as shown in screen 6570. The chatbot can provide different recommendations to the user depending on the user's answer. For example, if the user indicates “So far, so good,” the chatbot can provide a message such as “That's great. Remember: It's important to be kind to yourself when ‘the number’ isn't what you'd hoped for” with a link to a first article such as “Understanding if Your Diabetes Management Plan is Working.” If the user indicates “I′m struggling a bit,” the chatbot can provide a message such as “Hang in there. Developing a new habit isn't easy at first, but you've made a great start just by trying for this goal” with a link to a second article such as “How to Stay Motivated.”

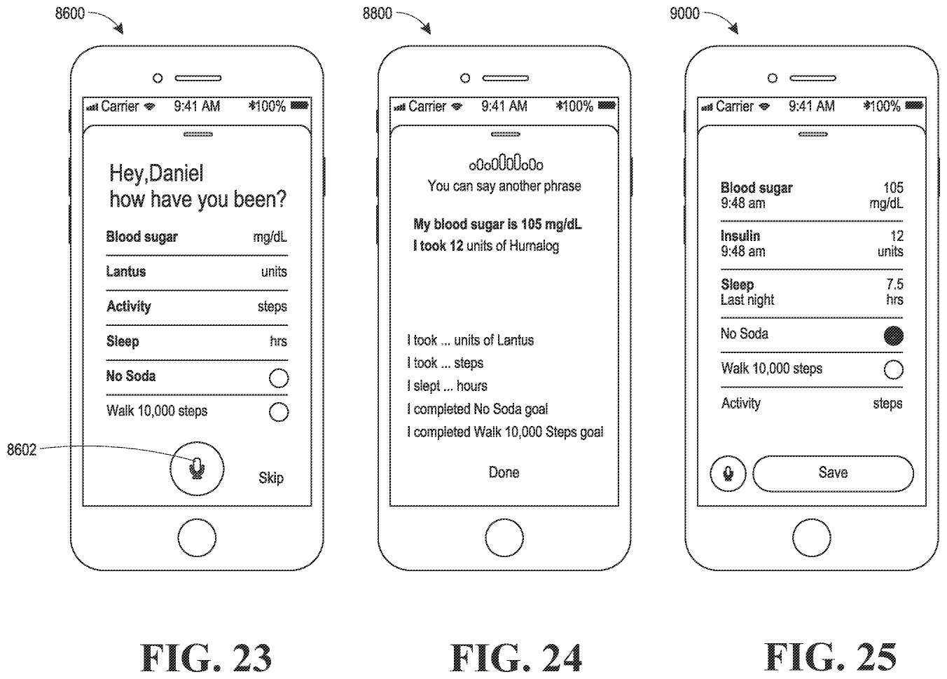

FIGS. 23, 24, and 25 are example screen captures of a logging module of a user interface of an IDM system according to an embodiment. FIG. 23 illustrates an example screen 8600 of a logging module. As shown, the screen 8600 includes a prompt asking the user, “Hey, Daniel, how have you been?” Following the prompts, the screen 8600 includes one or more potential data entry sources. For example, the screen 8600 includes data entry sources for blood sugar, Lantus® (a diabetes medication), activity, sleep, no soda, and walk 10,000 steps. Accordingly, the screen 8600 provides a simple method by which the user can enter data in each of these categories. Other categories may be included in other embodiments. Not all categories need be included in all embodiments.

As shown, the data entry categories can relate to various information pertinent to diabetes care. For example, data entry sources or categories can be included for various things such as physical measurements related to diabetes care such as blood sugar measurements, dosing information for medications taken related to diabetes (such as insulin and others), activity information such as a number of steps or number of minutes performing physical activity, number of hours slept, etc. Additionally data entry sources or categories can include items related to goals. For example, as illustrated, data entry sources or categories for “no soda” and “walk 10,000 steps,” goals described previously above in relation to the goals module, can be included.

The user can enter data for any of the data categories by selecting the data category on the screen 8600. Additional data categories may be available by scrolling down. The screen 8600 also includes a voice data entry button 8602 that the user can select to enter data vocally. Selecting the voice data entry by an 8602 may allow the user to speak the data that the user wishes to enter into the logging module. The logging module will then input the user's natural language and record the entered data as a voice file. The screen 8600 also includes a voice data entry button 8602 that the user can select to enter data vocally. Selecting the voice data entry button 8602 may allow the user to speak the data that the user wishes to enter into the logging module, and the logging module will parse the natural language and record the data.

FIG. 24 illustrates an example screen 8800 that can be displayed to the user after speaking one of the sample logging phrases. As shown, the user has spoken “my blood sugar is 105 mg/dl” and “I took 12 units of Humalog.” additional sample logging phrases are still displayed to the user providing additional prompts for logging data. Further, the screen 8800 can prompt the user to enter additional information by saying “you can say another phrase as shown.” As shown in FIG. 24, as the user enters data through the logging prompts the logging module transcribes the user spoken data onto the screen. This can allow the user to verify that the spoken data has been transcribed correctly. When the user selects done, each of the spoken data entries can be saved did IDM system for future use.

FIG. 25 illustrates an example screen 9000 that can be shown after data has been entered. The data may have been entered manually, for example by typing, or vocally by speaking as shown in the preceding examples. The screen 9000 presents the user with the data so that the user can verify and save the data.

FIG. 26 is an example screen capture of a data module of a user interface of an IDM system according to an embodiment. The data module can be configured to provide contextualized insights on the data screen based on the information available. Such information can include data entered by the user, for example, the logging module, or other data known the IDM system. Further, the data module can provide contextualized insights related to the data or content that the user is currently looking at. For example, if the user is looking at data, the data module will give contextual insights based on the data. As another example, if the user is looking at curriculum (for example, in the learn module), the user can be presented with contextual insights based on the curriculum. The data module can be configured to analyze combinations of data sets to produce insights, and then engage with the user with the chatbot, notifications, or other prompts. In some embodiments, example data sets include insulin, blood sugar, steps, and sleep. Analysis of the data sets can be defined by rules (as described above) or other algorithms.

FIG. 26 illustrates an example screen 9100 that includes a contextualized insight as described above. In this example, the user is viewing data related to blood sugar. A graph depicts the user's blood sugar over the past week. The data module can analyze this data while the user is viewing it and provide a contextualized insight in the form of a comment or notification. As shown, the screen 9100 displays “Your blood sugar has been out of target range for the last four Wednesdays. Are you doing something different? Let's chat about it.” In this case, this system has analyzed the blood sugar data set and determined that the user is consistently out of target range on Wednesdays, and then has engaged the user to determine why this may be. The screen 9100 includes a prompt that would allow the user to enter the chatbot so as to engage with the system through natural language, either entered on a keyboard or spoken vocally.

The screen 9100 also includes a menu with icons that take the user to different modules of the IDM system. For example, the menu includes the icon 4304 for accessing the data module, the icon 4305 for accessing the learn module, the icon 4306 for accessing the goals module, the icon 4307 for accessing the chatbot module, and the icon 4308 for entering user data with a logging module. These icons may also appear on the home screen 4300, as shown in FIG. 16, described above. These icons can allow for quick and easy access to other modules directly from within the learn module.

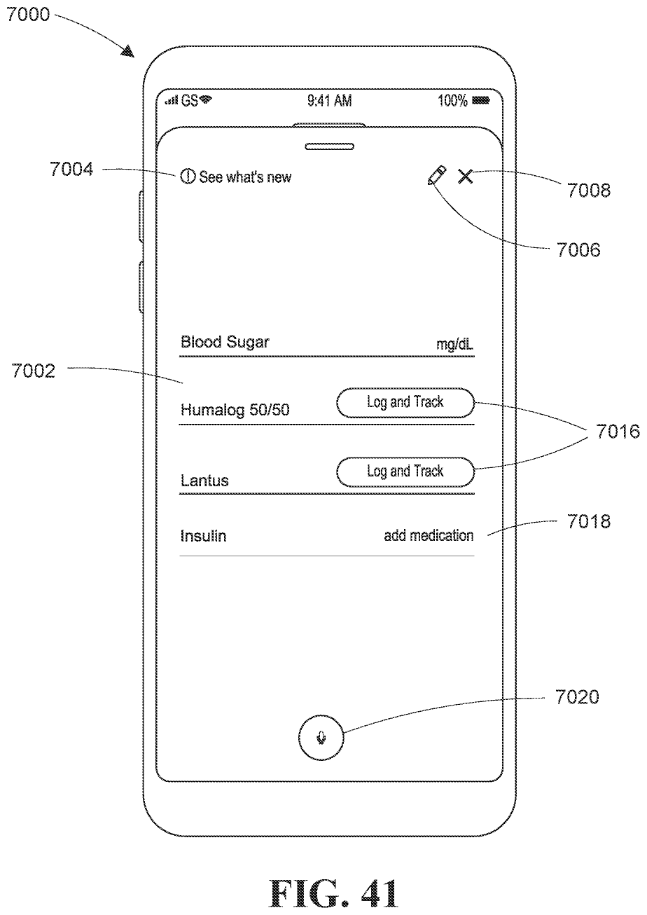

FIGS. 41-52 are example screen captures of a logging module of a user interface according to another embodiment. FIG. 41 illustrates an example screen 7000 of a logging module. The screen 7000 includes one or more data entry sources or categories 7002. For example, the screen 7000 includes data entry categories 7002 for blood sugar, Humalog® (a diabetes medication), Lantus® (a diabetes medication), and insulin. Accordingly, the screen 7000 provides a simple method by which the user can enter data in each of these categories. Other categories may be included in other embodiments. Not all categories need be included in all embodiments.

In some embodiments, the screen 7000 can include a new content button 7004 that can be selected by a user to show updates regarding the logging module. As shown in FIG. 41 the button 7004 includes the text “see what's new.” In some embodiments, selection of the button 7004 will open one or more new screens or cards showing updates regarding the logging module.

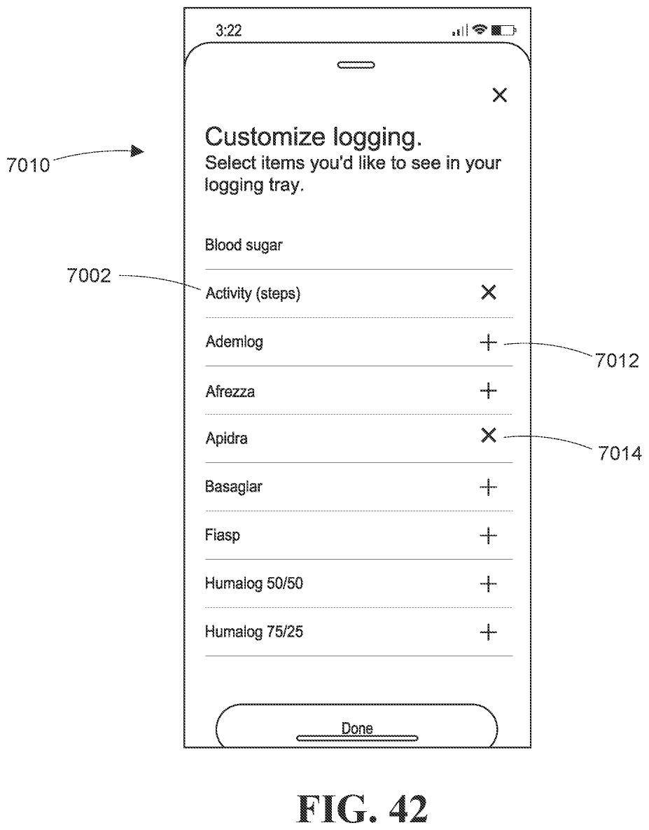

In some embodiments, the screen 7000 can include a customization button 7006. Selection of the customization button 7006 can open a customize view that can be used to add, remove, and/or modify data entry categories on the screen 7000. In some embodiments, the customization button 7006 can be in the form of a pencil icon. An example of a screen 7010 showing a customize view is illustrated in FIG. 42. As shown in FIG. 42, the customize view can include a number of potential data entry categories 7002. Each data entry category can be associated with a button 7012 for adding the data entry category to the screen 7000, for example in the form of a “+” icon, and/or a button 7014 for removing the data entry category from the screen 7000, for example, in the shape of an “X” icon.

With further reference to FIG. 41, in some embodiments, the screen 7000 can include a close button 7008. Selection of the close button can close the screen 7000. In some embodiments, the close button can be in the form of an “X” icon.

As shown, the data entry categories 7002 can relate to various information pertinent to diabetes care. For example, data entry sources or categories can be included for various things such as physical measurements related to diabetes care such as blood sugar measurements and dosing information for medications taken related to diabetes (such as insulin and others). In some embodiments, a data entry category 7002 can include a log and track button 7016 that can be selected to log and track information related to a medication. For example, screen 7000 includes a log and track button 7016 that can be selected for logging and tracking data related to Humalog® and a log and track button 7016 that can be selected for logging and tracking data related to Lantus®. Screen 7000 also includes an option to add an additional insulin medication for logging and tracking. In some embodiments, selection of the log and track button can redirect the user to a separate log and track screen for view and/or enter additional data related to medication. In some embodiments, the screen 7000 can include an add medication button 7018 to add another medication to the screen 7000 without having to first navigate to the customize view, such as the customize view shown in FIG. 42.

In some embodiments, data entry sources or categories can include activity information such as a number of steps or number of minutes performing physical activity, number of hours slept, etc. Additionally data entry sources or categories can include items related to goals. For example, data entry sources or categories for “no soda” and “walk 10,000 steps,” goals described previously above in relation to the goals module, can be included.

The user can enter data for any of the data categories by selecting the data category on the screen 7000. Additional data categories may be available by scrolling down. As shown in FIG. 41, the screen 7000 can also include a voice data entry button 7020 that the user can select to enter data vocally. Selecting the voice data entry button 7020 may allow the user to speak the data that the user wishes to enter into the logging module. The logging module will then input the user's natural language and record the entered data as a voice file.

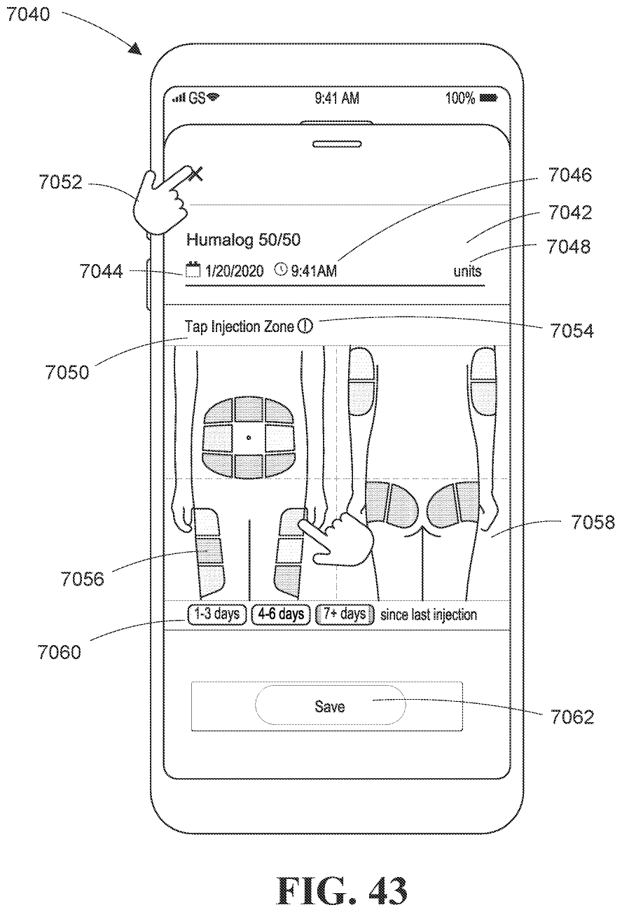

FIG. 43 illustrates an example screen 7040 that can be displayed to the user after the user selects the log and track option for an injectable medication. In the example screen 7040, the injectable medication is an injectable insulin medication, Humalog® 50/50. In some embodiments, the screen 7040 can include a medication logging section 7042 at which the user can enter information relating to an administered dose of medication. For example, the user can enter information relating to a date, time, and/or amount of medication taken in a particular dose. The medication logging section can include a date field 7044, a time field 7046, and a dosage units field 7048 for entering a number of units of the medication administered in the particular dose. In some embodiments, the present date and time may be displayed to allow a user to input information regarding a dose performed contemporaneously with data entry. In some embodiments, the present date and time are used by default, but can be changed through additional input by the user. In some embodiments, an entry for a date, time, and/or number of units can be edited at a later time, for example, my adding, changing, or deleting values. In some embodiments, values that are deleted or changed may be saved, but are no longer displayed to the user.

In some embodiments, the screen 7040 can include a close button 7052. Selection of the close button 7052 can close the screen 7040. In some embodiments, selection of the close button can redirect the user to screen 7000 as shown in FIG. 41. In some embodiments, the close button can be in the form of an “X” icon.

The screen 7040 can also include a site rotation section 7050. The site rotation section can provide a user with information regarding past injection sites using the medication. The site rotation section 7050 can also include options to allow a user to input information regarding injection site use. It can be beneficial for a user to rotate injection sites between injections, for example, to mitigate lipohypertrophy at the injection site. Lipohypertrophy can impact insulin absorption efficacy. In some instances, it may be beneficial to avoid using the same injection site for a particular period of time after a previous injection and/or for a particular number of injections after a previous rejection. In some instances, it may be beneficial to avoid using injection sites within a particular distance from a previous injection site, such as 1 finger width, 0.5 inches, 0.75 inches, 1 inch, or any other suitable distance, for a particular period of time after a previous injection and/or for a particular number of injections after a previous rejection. For example, it may be beneficial to avoid using a previous injection site and/or an area adjacent to a previous injection site for more than 3 days after the injection. It may be more beneficial to avoid using a previous injection site or area adjacent to the previous injection site for more than 6 days after the injection. In some embodiments, the site rotation section 7050 includes an information button 7054 that can be selected by a user to redirect the user to an article regarding site rotation.

The site rotation section 7050 also includes a diagram of at least a portion of the human body including a plurality of injection zones 7056. The injection zones 7056 are visual representations of segments of the body suitable for injection sites for the medication. For example, as shown in FIG. 43, the site rotation section 7050 illustrates injection zones 7056 around the abdomen, the thighs, the back of the arms, and the buttocks. In some embodiments, the injection zones 7056 can be dimensioned and/or shaped to facilitate ease in selection by a user using a finger to select an injection zone 7056 on a mobile device. In some embodiments, the injection zones 7056 can be further organized into regions 7058. For example, in FIG. 43, the injection zones 7056 are organized into four regions or quadrants 7058 including a first region 7058 including the abdomen, a second region 7058 including the thighs, a third region 7058 including the back of the arms, and a fourth region 7058 including the buttocks. In some embodiments, the dimensions and shape of the injection zones 7056 within each region 7058 are based on the total surface area available for injection within the region 7058 and the desired number of injection zones 7056 within the region 7058.

As shown in FIG. 43, in some embodiments, the screen 7040 can provide information regarding the injection history and/or suitability of use of a particular injection zone. For example, the screen 7040 can indicate for each injection zone 7056 a period of time since an injection was performed within the injection zone 7056. In some embodiments, the period of time since an injection was performed within an injection zone 7056 can be determined based on injection information provided by the user. For example, the user can enter the injection zone 7056 of an injection site used for an injection when logging the date, time, and/or amount of a particular dosing event. The screen 7040 can indicate for each injection zone 7056 a period of time since the time entered by the user for the most recent injection in the injection zone 7056. For example, in some embodiments, the screen 7040 can display the injection zones 7056 in different colors, shades, and/or patterns based on the duration of time elapsed since a previous injection in the injection zone 7056. In some embodiments, an injection zone 7056 can be shown in red if a most recent injection in that injection zone 7056 was recorded between 1-3 days before a present time. In some embodiments, an injection zone 7056 can be shown in orange if a most recent injection in that injection zone 7056 was recorded in the between 4-6 days before a present time. In some embodiments, an injection zone 7056 can be shown in green if a most recent injection in that injection zone 7056 was recorded seven or more days before a present time. As shown in FIG. 43, the screen 7040 also includes a color and/or pattern key 7060 explaining the meaning of the colors and/or patterns used to display the statuses of the injection zones 7056. Although patterns are shown in FIG. 43, other indicators may be used to provide an indication to a user of a period of time since a previous injection was performed in a particular injection zone. For example, in some embodiments, the indicators can be in the form of numbers, letters, symbols, or any other suitable format.

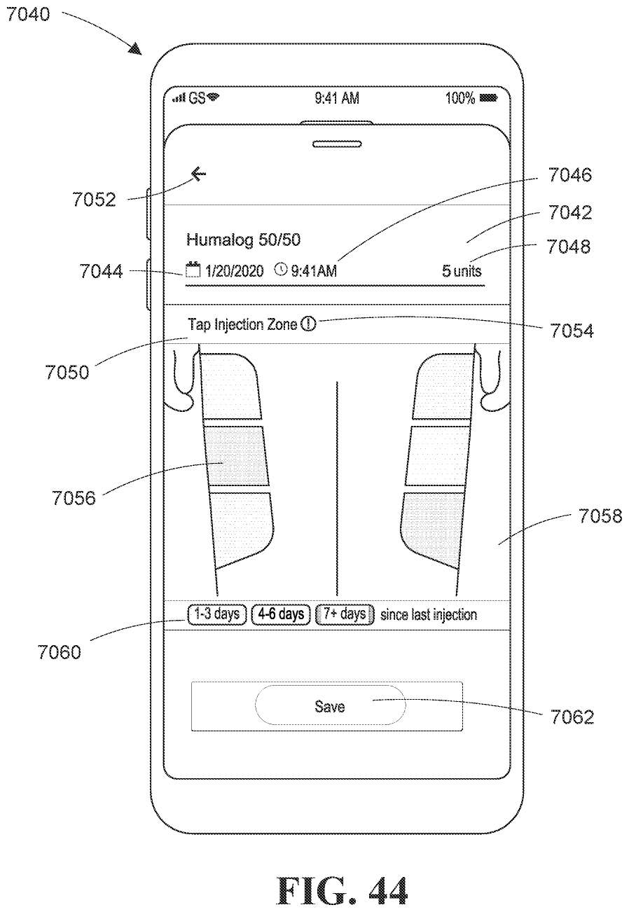

In some embodiments, an injection zone 7056 or a region 7058 of injection zones can be selected by a user on the screen 7040, for example, by tapping on the injection zone 7056 or the region 7058. Selection of the injection zone 7056 or the region 7058 can cause additional information regarding the injection zone 7056 or the region 7058 to be displayed. In some embodiments, selection of the injection zone 7056 or the region 7058 can cause a magnified or zoomed-in view of the injection zone 7056 or the region 7058 to be displayed. The magnified view can be displayed on the screen 7040 or on a separate screen. FIG. 44 illustrates an example of a magnified view of a region 7058 of injection zones including the thighs displayed on the screen 7040 after selection of the region 7058.

In some embodiments, an injection zone 7056 can be selected one the screen 7040 to record that the injection zone 7056 was used in an injection event performed by the user. The entry of the injection zone 7056 can be associated with the medication logging data entered by the user, such as the date, time, and/or amount of medication taken in a particular dose. In some embodiments, the injection zone can be selected either in the view shown in FIG. 43 or in the magnified view shown in FIG. 44 to record the injection zone 7056 used for an injection performed by the user.

As shown in FIGS. 43 and 44, the screen 7040 can include a save button 7062. The save button 7062 can be used to save data entered in the medication logging section 7042 and/or site rotation section 7050. In some embodiments, the save button 7062 can be a conditional save button. The save button 7062 can activate only when new data has been entered using the screen 7040. The save button 7062 may be inactive when no new data has been entered after opening the screen 7040 or after a previous save event. In some embodiments, the save button 7062 can include an indicator to indicate that the save button 7062 is inactive or active. For example, the save button 7062can be gray when inactive and orange when active.

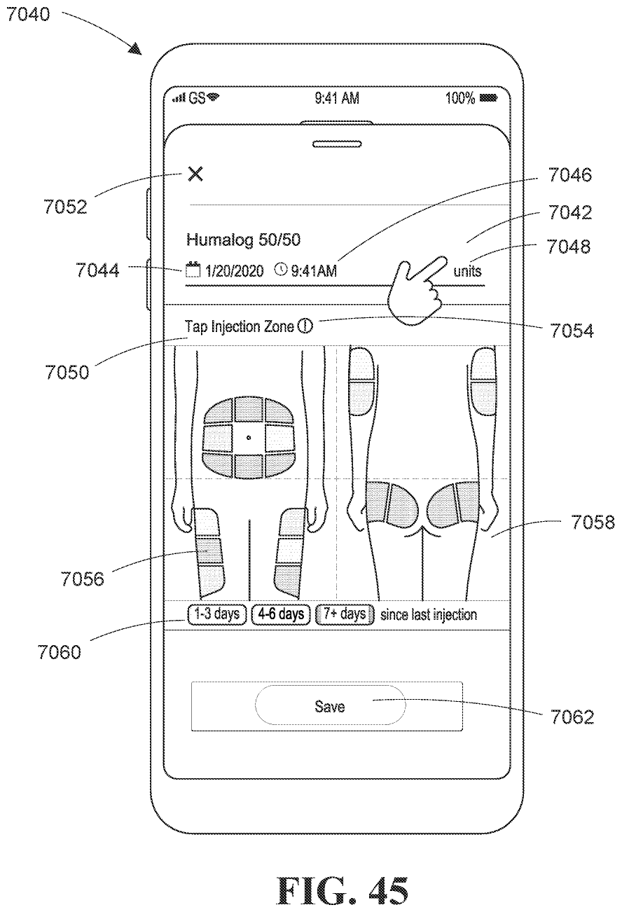

FIGS. 45-52 are example screen captures of the logging module depicting an example process for a user logging medication data and tracking an injection site for a particular dosing event.

FIG. 45 shows an example of screen 7040 during an initial step for logging medication data in the example process. As shown in FIG. 45, a user can start logging medication data by selecting the units field 7048. In some embodiments, the user may also select a date using the date field 7044 and/or a time of administration of a dosage of the medication using the time field 7046, for example, when entering information regarding a previously administered dose. In other embodiments, the present date and time may be displayed to allow a user to input information regarding a dose performed contemporaneously with data entry. In some embodiments, the present date and time are used by default, but can be changed through additional input by the user.

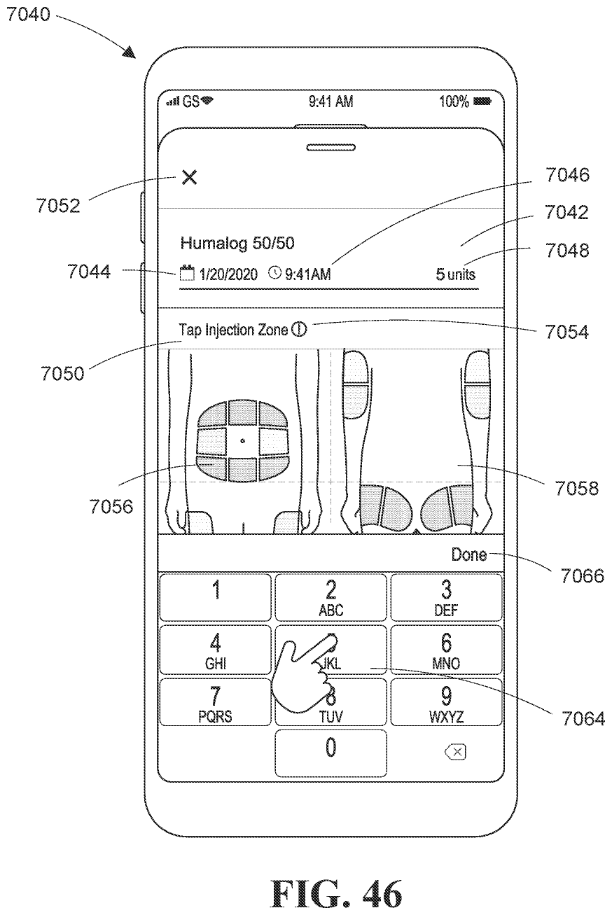

FIG. 46 shows an example of the screen 7040 after selection of the units field 7048. After selection of the units field 7048, a numeric tray 7064 can be displayed to the user to allow for entry of a number of units of medication for the dosing event. After selecting a number of units, the user can select a done button 7066 to close the numeric tray 7064.

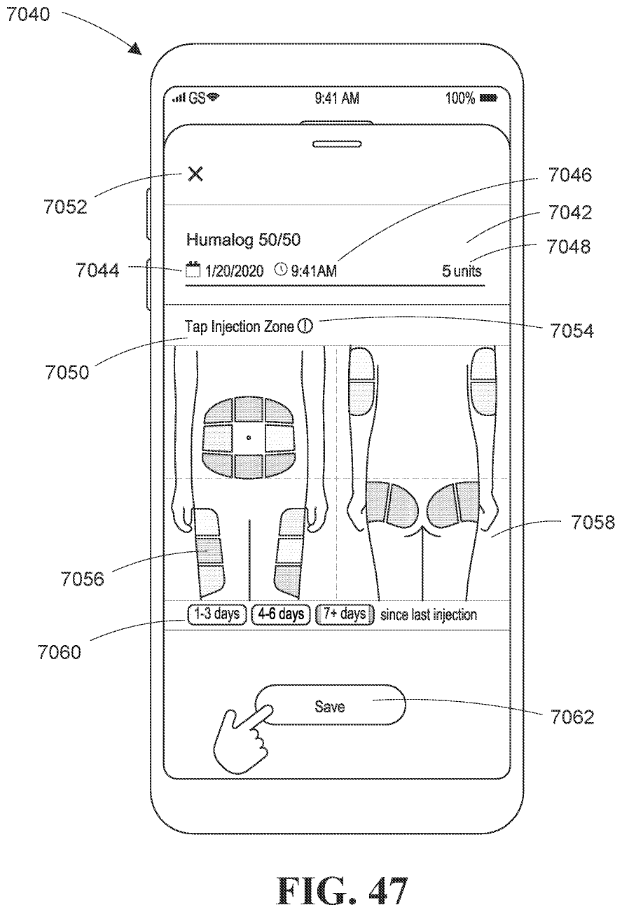

FIG. 47 shows an example of the screen 7040 after entry of a number of units and closing of the numeric tray 7064. As shown in FIG. 47, the save button 7062 is activated following the entry the number of units. The user can select the save button 7062 to save the medication logging data including the number of units, the date, and/or the time of the dosing event. Alternatively, the user can wait until after adding additional information to select the save button 7062.

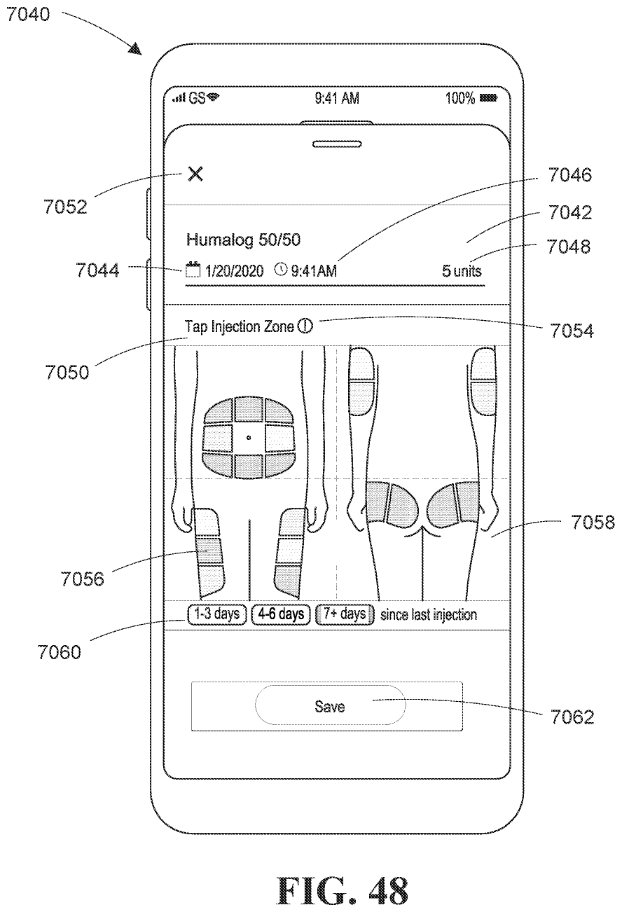

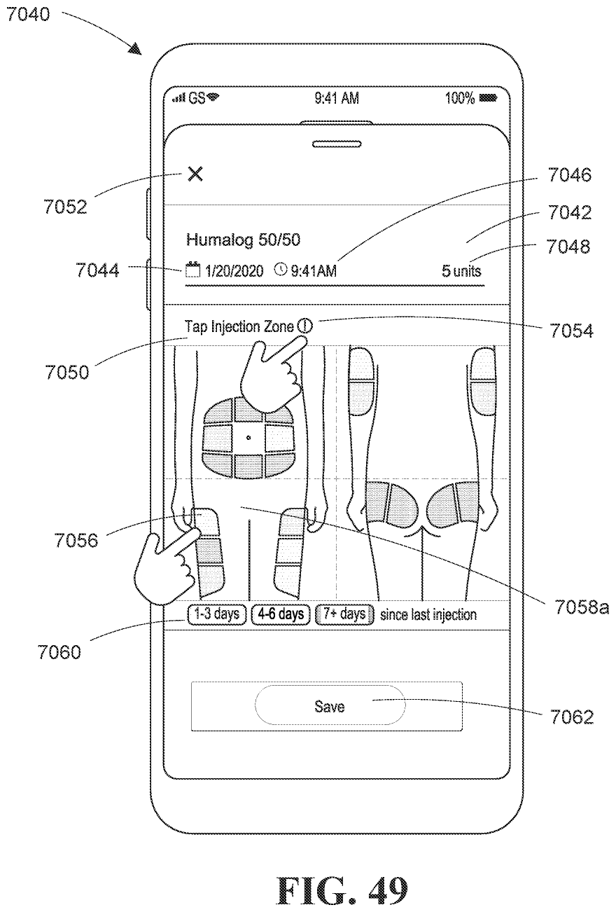

FIG. 48 shows an example of the screen 7040 after the logging data is saved by selecting the save button 7062. As shown in FIG. 48, the save button 7062 is inactive. After saving data, the user may choose to close the screen 7040 by pressing the close button or choose to enter injection site tracking information as shown in FIGS. 49-52. In some embodiments, the user may choose to enter injection site tracking information before saving data.

FIG. 49 shows an example of the screen 7040 during an initial step of tracking the injection site in the example process. The user can begin tracking the injection site by selecting an region 7058 of injection zones 7056 in the site rotation section 7052. As shown by the image of a hand in FIG. 49, the user is selecting a region 7058a including the injection zones 7056 located on the thighs. In some embodiments, the user can also optionally select the information button 7054 to redirect the user to an article regarding site rotation before or after selecting a region.

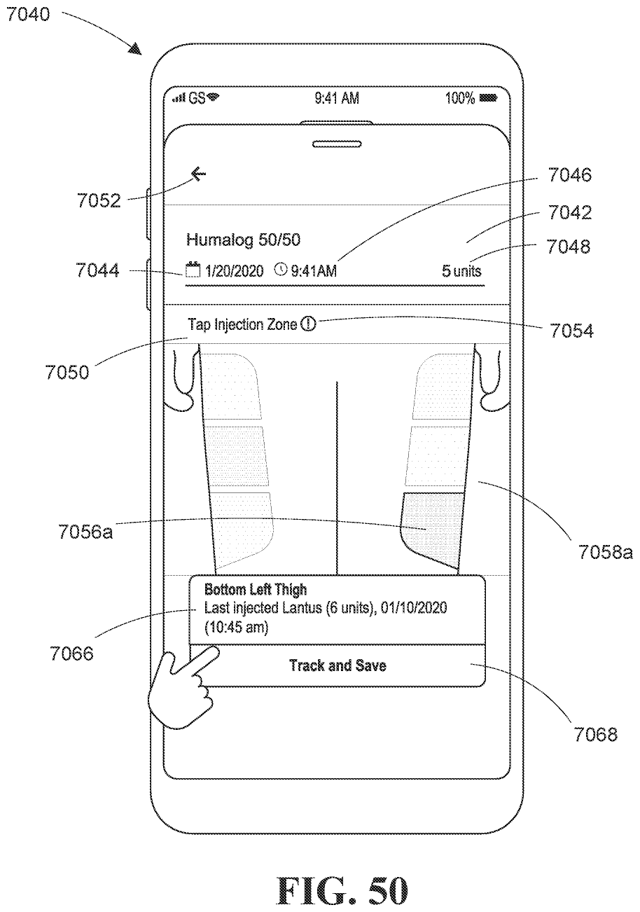

FIG. 50 shows an example of the screen 7040 after selecting the region 7058a of injection zones 7056 in FIG. 49. After selecting the region 7058, the user can select a particular injection zone 7056 for more information regarding that injection zone 7056. FIG. 50 shows a magnified or zoomed-in view of the selected region 7058a further showing the selection of a particular injection zone 7056. As shown in FIG. 50, after selection of a particular injection zone 7056, the screen 7040 can display information regarding the injection zone 7056. For example, as shown in FIG. 50, the screen can display the injection site name, the most recent injection date and time for the injection zone 7056a, the name of the medication used in the most recent injection, and/or the number of units of the most recent injection. In some embodiments, the information regarding the injection zone 7056 can be displayed in a mini-tray 7066 with a track and save button 7068. As shown in FIG. 50 an injection zone 7056a named “Bottom Left Thigh” is selected. The last injection was Lantus® in an amount of 6 units at 10:45 am on Jan. 10, 2020. The selected injection zone 7056a is shown in green indicating that the most recent injection 7 or more days prior to the present time. As shown in FIG. 50, the user can select the Track and Save button 7068 to record that that this injection zone 7056a has been used in the present dosing event. Selecting the track and save button 7068 will also save any other unsaved information. Alternatively, if the user does not wish to save the injection zone 7056a the user can tap a location outside of the mini-tray 7066 to close the mini-tray 7066.

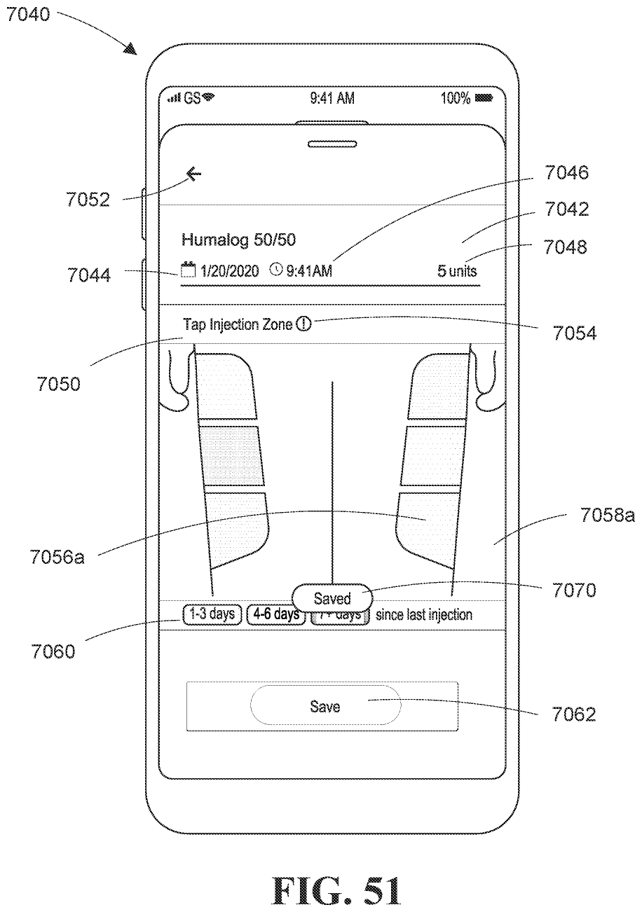

FIG. 51 shows an example of the screen 7040 after the injection zone 7056a for the bottom left thigh has selected and saved. As shown in FIG. 51 an indicator 7070 in the form of a capsule with the word “Saved” is present confirming that the tracking information was saved. In some embodiments, the indicator 7070 may appear for a predetermined period of time, such as several seconds, and then disappear. As shown in FIG. 51, the saved injection zone 7056a for the present dosing event is now shown in red to indicate that the most recent injection has occurred 3 or less days before the present time.

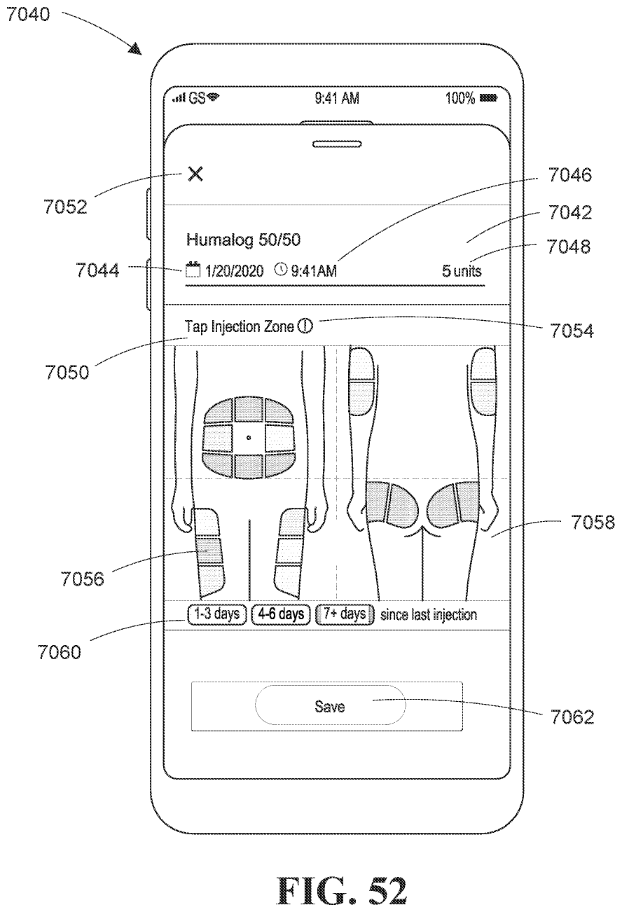

FIG. 52 shows an example of the screen 7040 after the indicator 7070 confirming that the tracking information was saved has disappeared. In some embodiments, when the indicator 7070 disappears, the screen 7040 can return to the full view of all of the injection zones 7056.

Although the process shown in FIGS. 49-52 depicts a user logging medication data in FIGS. 45-48 before entering tracking information in FIGS. 49-52, in some embodiments the user can enter tracking information before logging medication data. In some embodiments, the user may only log medication data without entering tracking information or only enter tracking information without logging the medication data.

### Example Implementing Systems

Implementations disclosed herein provide systems and methods for IDM systems and related devices or modules. One skilled in the art will recognize that these embodiments may be implemented in hardware, software, firmware, or any combination thereof. Those of skill would further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention. A software module may reside in random access memory (RAM), flash memory, ROM, EPROM, EEPROM, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. In other words, the processor and the storage medium may reside in an integrated circuit or be implemented as discrete components.

The functions described herein may be stored as one or more instructions on a processor-readable or computer-readable medium. The term “computer-readable medium” refers to any available medium that can be accessed by a computer or processor. By way of example, and not limitation, such a medium may comprise RAM, ROM, EEPROM, flash memory, CD-ROM or other optical disk storage, magnetic disk storage or other magnetic storage devices, or any other medium that can be used to store desired program code in the form of instructions or data structures and that can be accessed by a computer. Disk and disc, as used herein, includes compact disc (CD), laser disc, optical disc, digital versatile disc (DVD), floppy disk and Blu-ray® disc where disks usually reproduce data magnetically, while discs reproduce data optically with lasers. It should be noted that a computer-readable medium may be tangible and non-transitory. The term “computer-program product” refers to a computing device or processor in combination with code or instructions (e.g., a “program”) that may be executed, processed or computed by the computing device or processor. As used herein, the term “code” may refer to software, instructions, code or data that is/are executable by a computing device or processor.

Software or instructions may also be transmitted over a transmission medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of transmission medium.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component or directly connected to the second component. As used herein, the term “plurality” denotes two or more. For example, a plurality of components indicates two or more components.

The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

In the foregoing description, specific details are given to provide a thorough understanding of the examples. However, it will be understood by one of ordinary skill in the art that the examples may be practiced without these specific details. For example, electrical components/devices may be shown in block diagrams in order not to obscure the examples in unnecessary detail. In other instances, such components, other structures and techniques may be shown in detail to further explain the examples.

It is also noted that the examples may be described as a process, which is depicted as a flowchart, a flow diagram, a finite state diagram, a structure diagram, or a block diagram. Although a flowchart may describe the operations as a sequential process, many of the operations can be performed in parallel, or concurrently, and the process can be repeated. In addition, the order of the operations may be re-arranged. A process is terminated when its operations are completed. A process may correspond to a method, a function, a procedure, a subroutine, a subprogram, etc. When a process corresponds to a software function, its termination corresponds to a return of the function to the calling function or the main function.

The previous description of the disclosed implementations is provided to enable any person skilled in the art to make or use the present invention. Various modifications to these implementations will be readily apparent to those skilled in the art, and the generic principles defined herein may be applied to other implementations without departing from the spirit or scope of the invention. Thus, the present invention is not intended to be limited to the implementations shown herein but is to be accorded the widest scope consistent with the principles and novel features disclosed herein.

What is claimed is:

1. A method for tracking injection site information, comprising:
displaying, on a user interface, a plurality of injection zones, each of the plurality of injection zones representing a segment of the body suitable for medication injection and comprising a graphical indicator indicating a period of time since a most recent previous injection in the injection zone;
receiving, via the user interface, injection information relating to a new injection in a particular injection zone; and
updating a graphical indicator of the particular injection zone on the user interface to indicate a new date and time of injection to the particular injection zone.

2. The method of claim 1, further comprising storing the injection information in a user database.

3. The method of claim 1, further comprising modifying the graphical indicator of the particular injection zone from a first appearance to a second appearance when a period of time since the injection changes from between one and three days to between four and six days.

4. The method of any one of claim 3, further comprising modifying the graphical indicator of the particular injection zone from the second appearance to a third appearance when the period of time since the injection changes from between four and six days to seven or more days.

5. The method of claim 1, wherein updating the graphical indicator of the particular injection zone comprises modifying an appearance of the graphical indicator when receipt of the injection information is within a period of one and three days from the date and the time of the injection and the period of time since the most recent previous injection in the particular injection zone before the injection is more than three days.

6. The method of claim 1, wherein the graphical indicator of each injection zone comprises a color or a pattern indicating the period of time since the most recent previous injection in the injection zone.

7. The method of claim 1, wherein the injection information comprises a dosage amount.

8. The method of claim 7, further comprising associating the particular injection zone with the dosage amount.

9. The method of claim 1, wherein the plurality of injection zones are organized into a plurality of injection regions, wherein the injection zones within each injection region are shaped and dimensioned at least in part based on a total surface area available for injection within the injection region and a desired number of the injection zones within the injection region.

10. The method of claim 1, wherein the injection information comprises a selection of the particular injection zone.

11. The method of claim 10, further comprising displaying additional information related to the particular injection zone on the user interface in response to receiving the selection of the one of the particular injection zone.

12. The method of claim 1, wherein the injection information comprises the date and the time of the injection.

13. The method of claim 12, further comprising associating the particular injection zone with the date and time of the injection.

14. A system for tracking injection site information, comprising:
an interactive user interface configured to display and receive user information; and
a memory having instructions that when run on a processor will perform a method comprising:
displaying, on the user interface, a plurality of injection zones, each of the plurality of injection zones representing a segment of the body suitable for medication injection and comprising a graphical indicator indicating a period of time since a most recent previous injection in the injection zone;
receiving, via the user interface, injection information relating to a new injection in a particular injection zone; and
updating a graphical indicator of the particular injection zone on the user interface to indicate a new date and time of injection to the particular injection zone.

15. The system of claim 14, further comprising a user database configured to store the injection information.

16. The system of claim 14, wherein the memory comprises instructions that when run on the processor will perform a method comprising modifying the graphical indicator of the particular injection zone from a first appearance to a second appearance when a period of time since the injection changes from between one and three days to between four and six days.

17. The system of claim 16, wherein the memory comprises instructions that when run on the processor will perform a method comprising modifying the graphical indicator of the particular injection zone from the second appearance to a third appearance when the period of time since the injection changes from between four and six days to seven or more days.

18. The system of claim 14, wherein updating the graphical indicator of the particular injection zone comprises modifying an appearance of the graphical indicator when receipt of the injection information is within a period of one and three days from the date and the time of the injection and the period of time since the most recent previous injection in the particular injection zone before the injection is more than three days.

19. The system of claim 14, wherein the graphical indicator of each injection zone comprises a color or a pattern indicating the period of time since the most recent previous injection in the injection zone.

20. The system of claim 14, wherein the injection information comprises a dosage amount.

21. The system of claim 20, wherein the memory comprises instructions that when run on the processor will perform a method comprising associating the particular injection zone with the dosage amount.

22. The system of claim 14, wherein the plurality of injection zones are organized into a plurality of injection regions, wherein the injection zones within each injection region are shaped and dimensioned at least in part based on a total surface area available for injection within the injection region and a desired number of the injection zones within the injection region.

23. The system of claim 14, wherein the injection information comprises a selection of the particular injection zone.

24. The system of claim 23, wherein the memory comprises instructions that when run on the processor will perform a method comprising displaying additional information related to the particular injection zone on the user interface in response to receiving the selection of the particular injection zone.

25. The system of claim 14, wherein the injection information comprises the date and the time of the injection.

26. The system of claim 25, further comprising associating the particular injection zone with the date and time of the injection.
